(19) United States
(12) Patent Application Publication
(10) Pub. No.: US 2023/0408508 A1
(43) Pub. Date: Dec. 21, 2023

(54) SYSTEM AND METHOD FOR DIAGNOSIS OF ORAL DISEASE

(71) Applicant: The University of Birmingham, Birmingham (GB)

(72) Inventors: Melissa GRANT, Birmingham (GB); Tim ALBRECHT, Birmingham (GB); Oliver IRVING, Birmingham (GB)

(21) Appl. No.: 18/035,124

(22) PCT Filed: Nov. 4, 2021
(86) PCT No.: PCT/GB2021/052870

§ 371 Date: May 3, 2023

(30) Foreign Application Priority Data
Nov. 4, 2020 (GB) 2017465.2
Mar. 25, 2021 (GB) 2104219.7

(51) Int. Cl. G01N 33/543; B01L 3/02

(52) U.S. Cl. CPC G01N 33/5438; B01L 3/022; G01N 2800/18; B01L 2400/0406; B01L 2300/0645; B01L 2300/0838

(57) ABSTRACT

The present invention relates to a system and a device for use in the diagnosis of an oral disease. Methods, kits and compositions for use in the diagnosis of oral disease are also provided. More particularly, the invention relates to a system comprising a probe which is configured to collect a fluid sample from the oral cavity of a subject, and a detector which is configured to detect in the fluid sample the presence and/or concentration of an analyte which is indicative of the oral disease.

The present invention relates to a system and a device for use in the diagnosis of an oral disease. Methods, kits and compositions for use in the diagnosis of oral disease are also provided. More particularly, the invention relates to a system comprising a probe which is configured to collect a fluid sample from the oral cavity of a subject, and a detector which is configured to detect in the fluid sample the presence and/or concentration of an analyte which is indicative of the oral disease.

Periodontal disease, also known as gum disease, is initiated by a build-up of plaque on the teeth and furthered by the subsequent immune response. The early stage of the disease is known as gingivitis, the symptoms of which include inflammation of the gums, which may bleed after brushing. This inflammation is usually a response to bacterial biofilms or plaques which have formed on teeth. If left untreated, gingivitis can progress to a more serious condition known as periodontitis, the symptoms of which may include loose teeth and gum abscesses.

Serious cases of periodontitis can lead to the loss of teeth and damage to the jaw bone and periodontal ligament, and can therefore have a significant impact on quality of life. Periodontal disease has also been associated with a number of other conditions including lung infections, cardiovascular disease and premature labour.

Periodontal disease is the leading cause of tooth loss in adults, and is believed to affect ˜10% of the population worldwide, and is particularly prevalent in people over the age of 65.

To diagnose periodontal disease, a dentist will typically look for a number of indicators including tooth movement, sensitivity, gum bleeding, swelling and pocket depth. Large pocket depths around teeth are an indicator of gum disease. However, pocket depth is only a secondary indicator in that it indirectly indicates the presence of disease. Furthermore, the depth of the pocket does not provide any information about the progression of the disease, and thus what type of therapy is most appropriate.

WO2014/037924 describes analysis of biomarkers of gingivitis and periodontitis using Fourier Transform—tandem Mass Spectrometry (FT MS/MS). However, this type of analysis requires sophisticated equipment and specialist training and is therefore not available to the vast majority of dentists.

WO2019141525 and WO2019141547 describe the detection of biomarkers of periodontal disease using mass spectrometry, infrared or immunological assays. Mass spectrometry and infrared analysis again require expensive equipment and specialist training. Immunological assays are time consuming and can only be carried out by a person skilled in molecular biology techniques. Furthermore, assays such as ELISAs require numerous reagents including multiple antibodies.

The present invention has been devised with these issues in mind.

According to a first aspect of the invention there is provided a system for diagnosing an oral disease in a subject, wherein the system comprises:
a probe which is configured to collect a fluid sample from the oral cavity of the subject; and
a detector which is configured to detect in the fluid sample the presence and/or concentration of an analyte which is indicative of the oral disease.

The detector may be configured to receive the fluid sample from the probe.

In some embodiments, the probe and the detector are separate components.

In some embodiments, the detector is in fluid communication with the probe i.e. the detector is coupled to the probe. For example, the probe may be coupled to the detector via a conduit, e.g. a tube or capillary, which enables transfer of the fluid sample from the probe to the detector. The detector may be coupled to the probe permanently or semi-permanently, or temporarily e.g. only during transfer of the fluid sample from the probe to the detector.

In other embodiments, the probe is not coupled to the detector. In such embodiments, the fluid sample may be deposited (e.g. manually) from the probe into a portal, opening or receptacle, from which the fluid sample is transferred to the detector.

In some embodiments, the detector and the probe are integrated into a single device. For example, both the probe and the detector may be contained within the same housing.

According to a second aspect of the invention, there is provided a device for use in diagnosing an oral disease in a subject, the device comprising a probe which is configured to collect a fluid sample from the oral cavity of the subject.

In some embodiments, the device further comprises a detector which is configured to detect in the fluid sample the presence and/or concentration of an analyte which is indicative of the oral disease. Conveniently, the probe or the device may be hand-held.

The system and device of the invention enable both local fluid sampling from specific sites within the mouth and, optionally, integrated analysis. This provides the ability to detect analytes indicative of periodontal disease with local spatial resolution. For example, analyte concentrations may be mapped for each individual tooth, thereby providing site-specific determination of disease progression, and in turn enabling therapy to be targeted appropriately. In addition to the benefit of spatial resolution, the integration of the probe and detector into a single system or device conveniently enables in situ detection and/or quantification of analytes (e.g. biomarkers), which can be carried out by a dentist or dental hygienist, rather than having to send a sample to a laboratory for analysis.

### Detector

In some embodiments, the detector is configured to detect the presence and/or concentration of the analyte using resistive pulse sensing (RPS).

The inventors have recognised that RPS can be adopted for identifying analytes (e.g. biomarkers) indicative of oral disease in fluid samples taken from the oral cavity (e.g. in saliva and/or GCF). RPS is suitable for both detecting the presence of analytes and their abundance, and thus can be used to measure local analyte concentration. Since RPS provides an electrical signal, the results of RPS detection are rapidly provided. The use of RPS thus enables real-time detection of analytes, significantly reducing the time required to analyse a fluid sample compared to traditional assays, such as ELISA. Since the results are available quickly, patients can be diagnosed and treated within a single appointment. This enhances the patient experience and reduces cost through a more efficient use of time and resources. In addition, the cost of RPS analysis is low since few reagents are required, and can be carried out with less specialist knowledge and training compared to techniques such as mass spectrometry.

In some embodiments, the detector comprises a nanopore.

As is known in the art, resistive pulse sensors can be used as single-molecule detectors. Typically, resistive pulse sensors comprise a single, well-defined nanoscale pore embedded in a membrane which separates two electrolyte-filled compartments, each containing one electrode. The application of a voltage between the electrodes results in an ion current, which in turn leads to potential drops and local electric fields in the cell. Suitably strong electric fields can pull charged objects in solution toward and eventually through the nanopore. Because the nanopore normally constitutes the largest source of resistance in the cell, such a translocation event can cause a measurable ion current modulation, thereby enabling detection of molecules in solution.

In some embodiments the nanopore is a solid state nanopore. Aa solid state (i.e. chip-based) nanopore typically comprises a pore in a membrane., The membrane may be formed from a material such as Si₂N₄, SiO₂, graphene or MoS₂.

In some embodiments the nanopore is a biological nanopore, e.g. a nanopore formed within a protein. Biological nanopores may be embedded in a lipid bilayer.

Ideally, the nanopore is sized so as to allow the passage of a single analyte, or a single carrier molecule with one or more analytes bound thereto, through the pore at a time. It will therefore be appreciated that the size of the nanopore may be selected by the skilled person in accordance with the size of the analyte and/or the size of the carrier molecule. For example, the pore size may be comparable to, or slightly larger than, the maximum dimension of the analyte.

In some embodiments, the nanopore may be at least 5, at least 10, at least 20, at least 30, at least 50, at least 70, at least 100 or at least 130 nm in diameter. In some embodiments the nanopore is no more than 200 nm, no more than 150 nm, no more than 100 nm in diameter, no more than 50 nm, no more than 40 nm, or no more than 30 nm in diameter. Suitably, the nanopore may be from 5 to 100, from 10 to 50, from 15 to 40 or from 20 to 30 nm in diameter. It will be appreciated that nanopores are not necessarily circular. As such, the “diameter” of the nanopore in this context refers to the average dimension of the pore. It will further be appreciated that references to the diameter of the nanopore refer to the internal diameter.

The nanopore may be provided within a nanopipette. Thus, in some embodiments the detector comprises a nanopipette.

As is known in the art, nanopipettes are a class of nanopores which can be used for the detection and analysis of single molecules in solution. Nanopipettes can be easily fabricated with highly controlled pore sizes, making them a cost effective alternative to traditional solid state nanopores. The detection and analysis of a single molecule using a nanopipette relies on resistive pulse sensing. For this, the nanopipette is filled with and the tip immersed in an electrolyte and a voltage is applied between an electrode in side and an electrode outside of the nanopipette to generate an electric field at the tip. This field drives the molecule of interest through the nanopipette pore, resulting in a detectable pulse.

The nanopipette may be formed from any suitable material, such as a metal, polymer, glass, quartz, organic materials (e.g. graphene) or inorganic materials (e.g. boron nitride). In some embodiments, the nanopipette is made from glass or quartz.

Nanopipettes may be fabricated using methods which will be known to those skilled in the art, such as those described herein. Typically, nanopipettes are pulled from capillaries (e.g. quartz) using mechanical pipette pullers.

In some embodiments, the device comprises a double-barrel nanopipette, also known as a theta pipette. As is known in the art, a double-barrel nanopipette comprises two channels terminating in adjacent nanopores at the tip of the nanopipette, which are separated by a gap of approximately 20 nm (Cadinu et al., Nano Letters 2017, 17, 6376; Cadinu et al., Nano Letters 2018, 18, 2738). Translocation of an analyte takes place from one channel to the other.

A double barrel nanopipette may function to collect the fluid sample, and as the detector for detecting the presence and/or concentration of an analyte in the fluid. For example, the double-barrel nanopipette may comprise a first barrel (or channel) and a second barrel (or channel). The first channel may be configured to collect a fluid sample from the subject (e.g. from the gingival crevice), for example by means of electroosmosis. Translocation of an analyte present in the fluid sample from the first channel to the second channel generates a detectable signal.

The first channel may comprise a first electrode. The second channel may comprise a second electrode. In use, e.g. after insertion of the nanopipette into the mouth of a subject, application of a voltage to the first electrode relative to the second electrode may be used to drive a fluid sample into the first channel by electroosmotic flow. Subsequently, applying a positive bias to the second electrode relative to the first electrode may be used to translocate analytes present in the fluid sample from the first channel to the second channel.

Translocation of the analytes from the first channel to the second may be carried out after removal of the nanopipette from the mouth of the subject.

The fluid sample may be incubated in the first channel with a carrier molecule functionalised with a capture moiety which is capable of specifically binding to a target analyte (e.g. a biomarker) indicative of an oral disease which may be present in the fluid sample. Translocation of the target analyte (e.g. biomarker) bound to the carrier molecule from the first channel to the second channel would generate a detectable signal which is distinguishable from the signal generated by carrier molecules not bound to target biomolecules.

It will be appreciated that the probe must be suitably sized and shaped for collecting a fluid sample from the oral cavity of the subject. It will therefore be appreciated that “configured to collect a fluid sample from the oral cavity of the subject” means that the probe is sized and shaped so as to enable sampling from small and/or difficult-to-reach locations. The size and shape of the probe may therefore depend on a number of factors, including the species and/or age of the subject. Suitably, the probe may comprise a curved or bent head. This facilitates the collection of fluid samples from inside the mouth.

At the terminus of the head, the probe may be provided with a tip which is configured for collecting a fluid sample. Fluid collection may be achieved by any suitable means, including suction, electroosmosis or capillary action. In some embodiments, the tip comprises a capillary.

The tip of the probe may be configured for collecting a fluid sample from a periodontal pocket. As is known in the art, a periodontal pocket is a space between a tooth and the surrounding gum tissue, caused by the gum pulling away from the tooth. Formation of periodontal pockets is a sign of gum disease. The depth of the pocket is an indicator of the progression of the disease.

Thus, in some embodiments the tip is tapered. A narrow, tapered shape facilitates insertion of the tip into a periodontal pocket so that a sample of GCF or saliva may be obtained. At its widest point, the tip may have an outer diameter of from 0.3 mm to 2 mm, from 0.4 mm to 1.8 mm, from 0.4 mm to 1.5 mm, from 0.5 mm to 1.4 mm, from 0.6 mm to 1.3 mm, from 0.7 mm to 1.2 mm, from 0.8 mm to 1.1 mm or from 0.9 to 1.0 mm, e.g. approximately 1 mm.

At its narrowest point, the tip may have an outer diameter of from 10 to 300 nm, from 20 nm to 200 nm, from 30 to 180 nm, from 50 nm to 150 nm, from 60 nm to 120 nm or from 80 nm to 110 nm, for example approximately 100 nm.

At its narrowest point, the tip may have an internal diameter of from 2 nm to 200 nm, from 5 nm to 180 nm, from 10 nm to 150 nm, from 20 nm to 120 nm, or from 50 nm to 100 nm. In some embodiments, at its narrowest point the tip has an internal diameter of from about 10 to about 100 nm.

In some embodiments, the tip and/or the head of the probe is disposable. This helps to avoid contamination between subjects.

The probe, or a portion thereof (e.g. the head or tip) may be formed from any material that is suitable for collecting a fluid sample from the oral cavity of a subject. Suitable materials include metal, plastic, quartz, glass, organic materials (e.g. graphene) or inorganic materials (e.g. boron nitride), or a combination thereof.

In some embodiments, the probe or the device comprises, or is constituted by, a periodontal probe. A periodontal probe is an instrument commonly used in the field of dentistry, which is primarily used to measure pocket depths around a tooth. A periodontal probe typically has a curved or bent head formed from a thin strand of material (e.g. metal), usually of circular cross-section, having a narrow, tapered tip with distance markings thereon. Usually, a periodontal probe has a solid tip. However, in the context of the present invention, the periodontal probe is configured for collecting a fluid sample from the oral cavity of the subject. Accordingly, the tip of the periodontal probe is hollow, or has a capillary therethrough.

Thus, in some embodiments, the tip of the probe has one or more markings on an outer surface thereof. The markings may be provided at a distance of 1 mm, 2 mm, 3 mm, 4 mm, 5 mm, 6 mm, 7 mm, 8 mm, 9 mm and/or 10 mm from the terminus of the tip (i.e. the end through which the fluid sample enters the probe). The markings may, for example, be lines applied to the surface of the tip, or indentations created in the surface.

In a further aspect, the present invention provides a periodontal probe comprising a detector which is configured to detect the presence and/or concentration of an analyte in a fluid in the oral cavity of a subject. In some embodiments, the detector comprises a nanopore or a nanopipette.

In some embodiments, the tip of the probe (e.g. the periodontal probe) comprises or is constituted by a nanopipette. Thus, in some embodiments, the detector forms a part of, i.e. it is integrated into, the probe.

In some embodiments, the tip of the probe (e.g. the periodontal probe) is hollow and houses a nanopipette. In some embodiments, a first electrode may be located inside the nanopipette, and a second electrode may be located outside of the nanopipette, inside the tip. In some embodiments, the nanopipette is a double-barrel pipette.

The probe, or a device comprising the probe, may comprise a handle for operation of the probe.

The provision of a periodontal probe with an integrated analyte detector is especially beneficial. In addition to being hand-held and convenient to use, the fact that dentists are already highly familiar with the use of periodontal probes means that a periodontal probe having analytical capabilities will be easily adopted. In particular, a periodontal probe comprising a nanopipette will enable diseased or inflamed gum tissue to be identified with high spatial resolution (i.e. at the tooth level), and a diagnosis obtained in real time. The invention thus provides a faster, more convenient and more cost effective solution to the diagnosis of periodontal disease, and accurate determination of the disease state.

The presence and/or concentration of one or more analytes in the fluid sample may be indicative of an oral condition or disease in a subject. In some embodiments, the presence and/or concentration of one or more analytes is indicative of the progression of the oral disease.

The oral disease may be periodontal disease, which may also be referred to as gum disease. The periodontal disease may be gingivitis, mild periodontitis, moderate periodontitis, severe periodontitis or very severe periodontitis.

Gingivitis is classified according to the 2017 World Workshop classification system at a patient/case level the presence of bleeding to probe at ≥10% of sites in an otherwise intact periodontium, or one where any bone/attachment loss has arisen for reasons other than periodontitis. Localised gingivitis is 10-30% of bleeding sites and generalised is >30% of sites.

A patient is a periodontitis case in the context of clinical care if:
1 . Interdental clinical attachment loss (CAL) is detectable at non-adjacent ≥2 teeth, or
2 . Buccal or oral CAL ≥3 mm with pocketing >3 mm is detectable at ≥2 teeth

Periodontitis is then staged and graded as:
Stage 1 (mild) periodontitis is characterised by patients exhibiting <15% or <2 mm bone loss due to periodontitis.
Stage 2 (moderate) periodontitis is defined as bone loss up to the coronal third of the root, or with 3-4 mm attachment loss.
Stage 3 (severe) periodontitis is defined as bone loss in the mid third of the root, or with ≥5mm attachment loss and ≤4 teeth lost.
Stage 4 (very severe) periodontitis is defined as bone loss in the apical third of the root, or with ≥5 mm attachment loss and ≥5 teeth lost.

Furthermore, localised periodontitis refers to <30% of teeth involved and generalised is >30%. The “progression”, i.e. the extent or severity of the oral disease, will therefore be understood as referring to whether the subject has gingivitis, mild periodontitis or severe periodontitis.

The “progression”, i.e. the extent or severity of the oral disease, will therefore be understood as referring to whether the subject has gingivitis, or mild, moderate, severe or very severe periodontitis.

In some embodiments, the analyte is a biomarker. One or more biomarkers may be detected to diagnose the oral disease, and/or determine the progression of the disease. In some embodiments, a combination of biomarkers may be detected.

The concentration of a biomarker may be increased or decreased in a fluid sample taken from a subject suffering from an oral disease, relative to the concentration present in a fluid sample taken from a subject without an oral disease, or relative to a reference or threshold value. For example, the concentration of a biomarker may be increased or decreased in a fluid sample taken from a subject without oral disease, or with an oral disease in an earlier stage of progression (e.g. gingivitis) relative to the concentration present in a fluid sample taken from a subject with an oral disease in a later stage of progression (e.g. mild, moderate, severe or very severe periodontitis). Thus, the detection and/or quantification of biomarkers may be used to distinguish between a lack of oral disease, gingivitis, and different stages of periodontitis.

In some embodiments, the biomarker is a protein. The biomarker may be selected from the group consisting of haemoglobin alpha, haemoglobin beta, haemoglobin delta, elastase, carbonic anhydrase 1, plastin 1, transaldolase, S100 calcium binding protein A8 (S100-A8, also known as calgranulin A), S100 calcium binding protein A9 (S100-A9, also known as calgranulin B) or S100P, myosin-9, Alpha-1-acid glycoprotein (A1AGP), matrix metalloproteinase-9 (MMP-9), Peptidyl-prolyl cis-trans isomerase A, Haptoglobin-related protein, Alpha-N-acetylgalactosaminidase, NADPH oxidase, pyruvate kinase (PK), interleukin-1β, free light chain kappa, free light chain lambda, hepatocyte growth factor (HGF), keratin 4 (K4), profilin, catalase, choline transporter-like protein 2 derivative, titin isoform N2B and combinations thereof. Nȩdzi-Góra et al. (Cent. Eur. J Immunol. 2016; 41(2):2) observed significantly higher concentrations of elastase and MMP-9 in patients with periodontitis compared to healthy individuals, demonstrating the utility of these proteins as biochemical indicators of the severity of periodontitis. Victor et al. (J. Int. Oral Health, 2014; 6(6):67-71) found significantly elevated levels of MMP-9 among smokers with chronic periodontitis.

The device or system may further comprise a power supply, for example a battery or a connector for receiving mains power.

In some embodiments the system further comprises a controller (e.g. a computer). The controller may be configured to control the operation of the system in use. For example, the controller may be configured to control the voltage applied to the electrodes of the nanopipette. The controller may comprise a user interface. The user interface may enable an operator to input instructions and/or parameters, such as voltages or timings. Optionally, the user interface enables the user to observe the signal generated by translocation of molecules through the nanopore. The controller may comprise a memory for storing the generated signal.

According to a third aspect of the invention, there is provided a composition comprising carrier molecules for the detection of a target analyte, each carrier molecule being functionalised with a capture moiety which is capable of specifically binding to a target analyte. The target analyte may be a biomarker which is indicative of an oral disease.

The carrier molecules may be configured for use in the detection of target analytes using resistive pulse sensing. For example, the size and/or shape of the carrier molecules may be selected such that they are able to pass through a nanopore one by one. As is known in the art, the use of carrier molecules can facilitate the detection of target analytes using nanopores by increasing the mass of the subject molecule, thereby reducing the translocation speed and improving the signal-to-noise ratio.

Carrier molecules which are able to specifically bind a target analyte can be used to facilitate the detection of target analytes in a fluid using resistive pulse sensing. A change in the ion current signature detected upon translocation through the nanopore, as compared to the signature of the carrier molecule alone (i.e. in the absence of the target analyte), may be indicative of the formation of a carrier molecule-target analyte complex and thus the presence of the target analyte in the sample.

In some embodiments, the carrier molecule comprises at least two, at least three or at least four capture moieties. In some embodiments all of the capture moieties of a single carrier molecule are specific for the same target analyte. Alternatively, one, some or each of the capture moieties on a single carrier molecule may be specific to a different target analyte.

Alternatively, each carrier molecule may comprise a single capture moiety.

The carrier molecules may be functionalised with one or more capture moieties which are capable of specifically binding to any of the biomarkers identified herein.

In some embodiments, the carrier molecules comprise nucleic acids. The nucleic acids may be DNA, RNA, or a nucleic acid analogue, or a mixture thereof.

As used herein, a ‘nucleic acid analogue’ is understood to mean a structural analogue of DNA or RNA, designed to hybridise to complementary nucleic acid sequences. A nucleic acid analogue may be distinguished from DNA or RNA by its phosphate backbone, sugar groups, and/or nucleobases. Examples of nucleic acid analogues include, but are not limited to, threose nucleic acid, glycol nucleic acid, morpholino oligomers, peptide nucleic acids (PNA), locked nucleic acids “LNA”, 2′-O-methyl nucleic acids, 2′-fluoro nucleic acids, phosphorothioates, and metal phosphonates.

In some embodiments, the carrier molecules are formed partially or entirely from nucleic acids. In some embodiments, the carrier molecules are formed from DNA. In some embodiments, the carrier molecules may be oligonucleotides.

The carrier molecules may be formed from single-stranded nucleic acid or double-stranded nucleic acid (e.g. single- or double-stranded DNA), or a combination thereof. For example, while the majority of each carrier molecule may be formed from a double-stranded nucleic add, the carrier molecule may also comprise portions of single-stranded nucleic acid. In some embodiments the carrier molecules comprise a double stranded backbone and at least one single-stranded portion (i.e. an overhang) extending from the backbone.

Oligonucleotides having a desired sequence may be synthesised chemically or using standard molecular biology techniques which are known to those skilled in the art. For example, a desired sequence may be constructed by ligating portions of the sequence into a suitable plasmid or vector. Copies of the desired sequence may then be obtained using PCR amplification.

The skilled person will be familiar with methods of preparing carrier molecules, such as via DNA self-assembly, as shown in Loh et al., 2018, Anal Chem. 90, 14063-14071. Protein capture probes may be attached to a nucleic acid which forms the carrier molecules prior to assembly, using established ligation chemistry. Additionally or alternatively, carrier molecules may be prepared using enzymatic modification of double-stranded DNA, using methods known to those skilled in the art.

Each carrier molecule may be no more than about 100, no more than about 50, or no more than about 20 kilo bases (for a single-stranded carrier molecule) or kilo base-pairs (for a double-stranded carrier molecule) in length. In some embodiments, each carrier molecule is no more than about 15, no more than about 10, no more than about 8, no more than about 6 or no more than about 4 kilo bases (for a single-stranded carrier molecule) or kilo base-pairs (for a double-stranded carrier molecule) in length. The longer the carrier, the more capture moieties can be incorporated. However, extremely long carrier molecules are more likely to suffer from secondary effects, such as DNA knotting or crowding in solution.

The capture moiety may comprise any suitable molecule which is able to selectively bind to the target analyte. Examples of such molecules include single-stranded nucleic acids (e.g. comprising a sequence which is complementary to that of a target analyte), aptamers (nucleic acid or peptide aptamers), affimers, antibodies, proteins, molecularly imprinted polymers (MIPs) and nucleic acid-protein fusion molecules.

In some embodiments, the capture moiety is an antibody. The term “antibody” as used herein, includes antibody fragments (e.g. Fab fragments, F(ab′)2 fragments), polyclonal antibodies, monoclonal antibodies, chimeric antibodies, humanized antibodies, or single chain antibodies (e.g. single chain variable fragments (scFV)). The antibody may be a bispecific antibody, e.g. an antibody that has a first variable region that specifically binds to a first antigen and a second variable region that specifically binds to a second, different, antigen. Antibodies may be produced using standard techniques which are well-known to the skilled person. Antibody fragments may be produced by the modification of whole antibodies or synthesized de novo using known recombinant DNA methodologies.

In some embodiments, each carrier molecule comprises an identifier moiety, or “barcode”. The identifier moiety results in a unique signal upon translocation of the carrier molecule through a nanopore, thereby enabling carrier molecules comprising different identifier moieties to be distinguished from each other. Carrier molecules which are functionalised with capture moieties that are specific for the same target analyte may each be provided with the same identifier moiety. Carrier molecules functionalised with capture moieties for different target analytes may be provided with different identifier moieties. This enables several target analytes to be assayed at the same time (i.e. multiplexing).

An identifier moiety may be, for example, a nucleic acid structure.

The composition may be in the form of a solution or suspension of the carrier molecules in a suitable solvent or buffer (e.g. TE buffer).

According to a fourth aspect of the invention, there is provided a kit for diagnosing an oral disease in a subject, the kit comprising:
a device comprising a probe which is configured to collect a fluid sample from the oral cavity of the subject; and
instructions for use.

The device and/or the probe may be one as defined herein.

In some embodiments, the kit further comprises a detector which is configured to detect in the fluid sample the presence and/or concentration of an analyte which is indicative of the oral disease.

The kit may comprise a nanopipette. In some embodiments, the nanopipette may be provided separately from the probe. Thus, the kit may comprise a nanopipette for insertion into a tip of the probe. In some embodiments, the kit comprises a plurality of nanopipettes. This enables the nanopipette to be changed between each patient or each sample.

The kit may further comprise a composition as defined herein.

The kit may additionally comprise one or more reference samples. A signal generated by the reference sample be compared with a signal generated by the fluid sample obtained from the patient, thereby facilitating the detection of the presence and/or concentration of the target analyte(s), and thus a diagnosis.

According to a fifth aspect of the invention, there is provided a method of diagnosing an oral disease in a subject, the method comprising detecting the presence and/or concentration of a target analyte in a fluid sample obtained from the oral cavity of the subject. The presence or concentration of the target analyte (e.g. a biomarker), may be indicative of the oral disease.

The method may be carried out using a device, a probe or a system as defined herein. For example, the fluid sample may be obtained, or may have been previously obtained, from the oral cavity of the subject using a probe or a device as defined herein. In some embodiments, the probe is a periodontal probe having a hollow tip.

In some embodiments, resistive pulse sensing is used to detect the presence and/or concentration of the target analyte. Thus, the invention provides the use of resistive pulse sensing to diagnose an oral disease in a subject. In some embodiments, the method comprises using a nanopore or a nanopipette to detect the presence and/or concentration of the target analyte. Detect the presence and/or concentration of the target analyte may be carried out using a detector as defined herein.

In some embodiments, the method is carried out using a device comprising a probe and an integrated detector. For example, the method may be carried out using a probe having a hollow tip in which is housed a nanopipette. The integration of the probe and the detector into a single device conveniently provides a compact device for the diagnosis of oral disease.

In alternative embodiments, the method comprises transferring the fluid sample from the probe to a separate detector which is configured to detect the presence and/or concentration of the analyte e.g. using resistive pulse sensing. The detector may be as defined herein. The detector may comprise a nanopore or a nanopipette.

The method may comprise:
applying a voltage across the nanopore to effect translocation of any molecules present in the fluid sample through the nanopore; and
detecting a signal generated by the translocation of molecules through the nanopore.

The voltage applied across the nanopore may be from 0.05 to 10 volts, from 0.1 to 9 volts, from 0.5 to 8 volts, from 1 to 6 volts, from 2 to 5 volts or from 3 to 4 volts.

In some embodiments, the method further comprises contacting the fluid sample with carrier molecules, prior to detection. The carrier molecules may be functionalised with a capture moiety which is capable of specifically binding to the target analyte (e.g. a biomarker) which is indicative of the oral disease. Translocation through the nanopore of a carrier molecule bound to the target analyte (i.e. a carrier molecule-analyte complex) may result in a signal that is distinguishable from a signal generated by an unbound carrier molecule, thereby enabling detection of the target analyte in the fluid sample.

In some embodiments, the fluid sample is incubated with carrier molecules prior to translocation, wherein the carrier molecules are functionalised with a capture moiety which is capable of specifically binding to a biomarker of an oral disease.

The fluid sample may be incubated with the carrier molecules for a period of time sufficient to enable the capture moieties to bind to the target analyte, if present in the fluid sample. Incubation may be carried out fora period of time of from 5 s to 1 hour, from 10 s to 30 minutes, from 30 s to 15 minutes, from 1 minute to 10 minutes, or from 2 minutes to 5 minutes. Incubation may be carried out at a temperature of from 10 to 35° C., from 15 to 30° C., from 18 to 25° C. or from 20 to 22° C. Suitably, incubation is carried out at room temperature (e.g. about 20° C.).

The signal may be a current-time signal (i.e. showing the change in the electric current across the nanopore as a function of time). As is known to those skilled in the art, nanopore detection works by detecting changes to the electric current as molecules are translocated through the pore. The translocation of a molecule (e.g. a carrier molecule) through the nanopore produces an event signature which is characteristic of that molecule. For example, each carrier molecule will generally produce sub-structure (sub-events) within the event. The properties of these sub-events (duration, magnitude, charge or other signal characteristics) can be used to detect whether or not the carrier molecule is bound to the target analyte.

The signal detected may then be compared to a reference signal. By comparison to a reference signal, the presence of the target analyte in the fluid sample may be determined. The reference signal may be one generated using a fluid sample obtained from a healthy subject without oral disease, or using a fluid sample which is known to contain, or known not to contain, the target analyte. In some embodiments, the reference signal is a signal generated by the translocation of unbound carrier molecules through the nanopore. Alternatively, the reference signal may be obtained from the literature.

In some embodiments, comparison of the signal detected with a reference signal may be used to determine whether the concentration of the target analyte (i.e. biomarker) in the fluid sample is elevated or reduced, relative to the reference. An increase or a decrease in concentration of a particular target analyte may be indicative of disease and/or the stage of the disease.

Thus, detecting a concentration of the analyte, as used herein, includes determining a relative concentration with respect to a reference, as well as determining absolute concentration.

In some embodiments, the method comprises determining, or estimating, the absolute concentration of the target analyte, using the detected signal. For example, the ratio of “bound” vs. “unbound” sub-events for a given target allows for an estimation of the target analyte concentration.

The absolute concentration of the target analyte present in the fluid sample may then be compared with a threshold value in order to diagnose whether the subject is suffering from an oral disease, the type of disease and/or the extent of the disease. The threshold value may be one generated using a fluid sample obtained from a healthy subject without oral disease, or from a subject known to be suffering from gingivitis or periodontitis. Alternatively, the threshold value may be obtained from the literature.

In some embodiments, the method comprises detecting the presence and/or concentration of a target analyte in multiple (e.g. 2, 3, 4, 5, 6 or more) fluid samples obtained from the oral cavity of the subject. The fluid samples may be obtained from different sites within the oral cavity. This enables sites of inflammation or disease, or the progression of disease, to be mapped. Thus, in some embodiments the method comprises generating a map of sites of disease within the oral cavity of the subject. The map may indicate the severity of the disease at each site.

In some embodiments, the method comprises detecting the presence and/or concentration of multiple (e.g. two, three, four, five, six or more) target analytes within the fluid sample. For example, a set of biomarkers may be used to diagnose an oral disease in a subject, and/or determine the progression of the disease. For example, a panel of biomarkers may be selected for distinguishing between a healthy subject (i.e. lack of oral disease) and a subject with inflammation or gingivitis, between gingivitis and periodontitis, or for distinguishing between different states of periodontitis (e.g. between mild and moderate, or moderate and severe periodontitis).

In a further aspect of the invention, there is provided a method of diagnosing an oral disease in a subject, the method comprising:
transferring a fluid sample from a probe to a detector, wherein the fluid sample was previously collected from the oral cavity of the subject using the probe; and
using the detector, detecting in the fluid sample the presence and/or concentration of a target analyte which is indicative of the oral disease.

In yet a further aspect, the invention provides a method for detecting the presence and/or concentration of a target analyte in a fluid sample using resistive pulse sensing, wherein the target analyte is a biomarker of an oral disease.

The fluid sample may be one which was previously obtained from the oral cavity of a subject. In some embodiments, the method further comprises obtaining the fluid sample from the oral cavity of the subject.

The fluid sample may be obtained by any convenient means, for example using electroosmosis, suction or capillary action. In some embodiments, the fluid sample is obtained using a probe as defined herein.

In some embodiments in which the probe comprises a periodontal probe housing a nanopipette, for each fluid sample obtained (e.g. for each tooth or each GCF measurement), a new nanopipette may be provided. This will avoid cross-contamination between teeth and allow for sterilisation of the periodontal probe (in the absence of the nanopipette) between samples.

The methods of the invention may be used to determine whether a subject is suffering from gingivitis, mild periodontitis or severe periodontitis.

In some embodiments, the method further comprises treating a subject diagnosed as having an oral disease. Treatment may include one or more of: scaling; root planning; administration of antibiotics (e.g. oral or topical); surgery; regular dental cleaning (e.g. every 3 or 6 months); and use of mouthwash (e.g. daily).

The fluid sample may be saliva or gingival crevicular fluid (GCF).

The subject may be a human or a non-human mammal, such as a horse, cow, pig, goat, sheep, dog, cat or primate.

The invention also relates to a modular polynucleotide and to a method of making a modular polynucleotide. The invention also relates to a method and a kit for determining if one or more analyte(s) is/are present in a sample, and a method and kit for diagnosis of a medical condition, using a modular polynucleotide. The invention also extends to a method of treatment, and a method of determining the efficacy of a therapeutic agent.

Assays are an important aspect of investigative research as they enable one to measure the presence, amount and/or functional activity of an analyte. Each analyte of interest may require a different specialised technique or equipment to detect its presence. For example, a sample suspected of containing several different analytes may require the use of gas chromatography-mass spectrometry (GC-MS) or high pressure liquid chromatography -mass spectrometry (HPLC-MS) to detect analytes present at low concentrations; enzyme linked immunosorbent assays (ELISA) or immunofluorescence (IF) to detect analytes in the form of polypeptides or proteins; and polymerase chain reaction (PCR) or a microarray to detect nucleic acids. While each technique enables detection and/or quantification of specific types of analytes, each technique is often also expensive and/or time-consuming. There is therefore a need for a rapid and cheap technology for detecting several different analytes.

The inventors have surprisingly developed a novel method of creating a modular double-stranded polynucleotide having subunits (i.e. (im)mature conjugate subunits) each comprising a probe for binding an analyte. The modular polynucleotide can be used with a carrier-enhanced resistive pulse sensing technology (e.g. nanopore-based resistive pulse sensing or nanopipette-based resistive pulse sensing) to simultaneously detect several different analytes, including different types of analyte, in a high-throughput manner.

Thus, according to a sixth aspect of the invention, there is provided a method of making an immature conjugate subunit, the method comprising:
a) conjugating a (first) probe, for binding a (first) analyte, to a probe conjugation site of a (first) double-stranded polynucleotide, to create a (first) immature conjugate subunit.

In one embodiment, there is provided a method of making a mature conjugate subunit, the method comprising:
a) conjugating a (first) probe, for binding a (first) analyte, to a probe conjugation site of a (first) double-stranded polynucleotide, to create a (first) immature conjugate subunit; and
b) forming a (first) mature conjugate subunit by cleaving the double-stranded polynucleotide of the (first) immature conjugate subunit to form a first sticky end, or a first sticky end and/or a second sticky end.

The first sticky end and/or the second sticky end of the mature conjugate subunit may be complementary to a (first or second) sticky end of a further, separate mature conjugate subunit, or complementary to a (first or second) sticky end of a further, separate mature conjugate subunit to be formed from an immature conjugate subunit.

Each mature conjugate subunit may further comprise a double-stranded polynucleotide spacer. The spacer may be annealed to the double-stranded polynucleotide of the (first) mature conjugate subunit so as to create a polynucleotide backbone. Thus, the method according to the sixth aspect may further comprise the step of:
c) annealing the first sticky end or the second sticky end of the (first) mature conjugate subunit to a complementary sticky end of a (first) double-stranded polynucleotide spacer.

The invention may further comprise creating a modular polynucleotide comprising a double-stranded polynucleotide backbone having two or more mature conjugate subunits. Thus, the method according to the sixth aspect may further comprise:
repeating steps (a) to (b) or steps (a) to (c) to create a total of two or more mature conjugate subunits, wherein each of the two or more mature conjugate subunits has a (first or second) sticky end that is complementary with a (first or second) sticky end of a separate mature conjugate subunit; and
annealing the sticky ends of the two or more mature conjugate subunits to create a modular polynucleotide comprising a double-stranded polynucleotide backbone having two or more mature conjugate subunits.

The method of creating a modular polynucleotide may comprise performing a one-pot synthesis reaction. In other words, the cleaving and annealing steps may be performed simultaneously in a single reaction vessel. Thus, the modular polynucleotide may be created by placing multiple immature conjugate subunits in a single reaction vessel together with an exonuclease, a DNA ligase and a DNA polymerase, and optionally multiple double-stranded polynucleotide spacers.

The modular polynucleotide may comprise two or more, three or more, four or more, five or more, six or more, seven or more, eight or more, nine or more, 10 or more, 20 or more, 50 or more, 100 or more, 200 or more, 300 or more, 500 or more, 1000 or more, 3000 or more, 5000 or more, 10000 or more mature conjugate subunits. The modular polynucleotide may comprise between two and 10000 mature conjugate subunits, two and 5000 mature conjugate subunits, two and 1000 mature conjugate subunits, two and 500 mature conjugate subunits, two and 100 mature conjugate subunits, two and 50 mature conjugate subunits, or two and 10 mature conjugate subunits.

The method of the invention may ultimately comprise attaching a plurality of mature conjugate subunits (each comprising a probe) together in a modular fashion. The initial polynucleotide used to create the mature conjugate subunits may be a blunt-ended polynucleotide. Prior art methods of attaching blunt-ended polynucleotides together can only be achieved with polynucleotides that are at least 100 base pairs in length (and by using an exonuclease, a DNA polymerase and a DNA ligase). However, unlike the prior art, the method according to the invention can be used to attach several different blunt-ended polynucleotides together that are significantly shorter in length (e.g. blunt-ended polynucleotides that are between about 20 and about 100 base pairs in length or between about 34 and about 100 base pairs in length).

The inventors believe (but do not wish to be bound by the theory) that the attachment of a probe to the blunt-ended polynucleotide sterically impedes the activity of nucleases (e.g. exonucleases) that may be used to cleave short (i.e. less than 100 base pairs) blunt-ended double-stranded polynucleotides (e.g. immature conjugate subunits). They believe that nucleases (such as exonucleases, e.g. T5 exonuclease) are unable to navigate across the site at which the steric hindering agent is located (e.g. the probe), and thus get knocked off of the blunt-ended polynucleotide. Consequently, the method according to the invention enables the blunt end of a short double- stranded polynucleotide to be converted into “sticky ends” without converting the entire double-stranded polynucleotide into a single-stranded polynucleotide.

The method according to the invention is also advantageous because it enables probes for different analytes, different classes of analyte and the same analyte to be conjugated to a single polynucleotide backbone of a modular polynucleotide. Consequently, a modular polynucleotide according to the inventio can be used to perform multiplex, high-throughput analysis using a single technique (i.e. carrier- enhanced resistive pulse sensing, such as nanopore-based resistive pulse sensing or nanopipette-based resistive pulse sensing).

Furthermore, the method according to the invention enables each probe of a modular polynucleotide to be conjugated at a specific location within the polynucleotide backbone. Consequently, each disruption of the electrical current generated during use with a carrier-enhanced resistive pulse sensing technology can be attributed to a particular probe, and the specific characteristics of each disruption can be used to determine if a probe has bound an analyte or not.

Furthermore, the method can be used to easily modify a modular polynucleotide so as to alter the analyte(s) being detected due to the modular nature of the immature conjugate subunits, which may each comprise an optional double-stranded polynucleotide spacer.

The probe conjugation site may comprise a nucleotide sequence specific for a transferase enzyme. Thus, the probe conjugation site may comprise or consist of a CpG island (or a CpG thereof) or the nucleotide sequence TOGA. In one embodiment, the probe conjugation site comprises a nucleotide sequence specific for a methyl transferase enzyme (e.g. m.Taq1). Thus, the probe conjugation site may comprise the nucleotide sequence TOGA, or a CpG island (or a CpG thereof).

A double-stranded (im)mature conjugate subunit may be created by a method according to the invention.

Thus, according to a seventh aspect, there is provided an immature conjugate subunit comprising:
a probe, for binding an analyte, conjugated to a probe conjugation site of a double-stranded polynucleotide,
wherein the probe conjugation site comprises a nucleotide sequence specific for a transferase enzyme.

The polynucleotide of the double-stranded mature conjugate subunit may be between about 20 and about 100 base pairs/nucleotides in length, or between about 34 and about 100 base pairs/nucleotides in length.

The polynucleotide of the double-stranded mature conjugate subunit may comprise blunt ends (e.g. a first blunt end and/or a second blunt end).

The polynucleotide of the double-stranded mature conjugate subunit may comprise sticky ends (e.g. 5′-sticky ends or 3′-sticky ends). Thus the conjugate subunit may be referred to as a mature conjugate subunit.

A double-stranded modular polynucleotide may be created by a method according to the invention. For example, a modular polynucleotide may be created by annealing a plurality of mature conjugate subunits together. Each of the mature conjugate subunits may be created by a method according to the invention.

Thus, according to an eighth aspect, there is provided a double-stranded modular polynucleotide comprising:
a double-stranded polynucleotide backbone comprising a plurality of probe conjugation sites each site having a nucleotide sequence specific for a transferase enzyme, wherein each probe conjugation site is separated by at least 16 base pairs, and wherein at least one probe conjugation site is conjugated to a single probe for binding an analyte.

Of the plurality of probe conjugation sites, at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% of the sites may be conjugated to a probe. 100% of the probe conjugation sites may be conjugated to a probe.

The modular polynucleotide may comprise different classes of probe, which are capable of binding to the same analyte or different analytes. The modular polynucleotide may comprise two or more different classes of probe, which are capable of binding to the same analyte or a different analyte.

The (im)mature conjugate subunit according to the seventh aspect may be used to form a modular polynucleotide according to the eighth of invention or a modular polynucleotide made by a method according to the invention. A single modular polynucleotide according to the invention may be used to perform multiplex, high- throughput analysis using a carrier-enhanced resistive pulse sensing technology to simultaneously detect different analytes using different probes.

The method according to the invention is also advantageous because it can be used to create a modular polynucleotide comprising a plurality of identical probes. None, some or all of the probes of such a modular polynucleotide may be capable of binding to relevant analyte molecules in a sample. Thus, such a modular polynucleotide can be used to improve the signal-to-noise ratio and/or reduce the total analysis time. The modular polynucleotide can also be used to provide quantitative information about analyte binding (analyte concentration in a sample).

The probe conjugation site referred to herein may comprise a nucleotide sequence specific for a transferase enzyme (e.g. a methyl transferase enzyme). Thus, the probe conjugation site may comprise or consist of a CpG island (or a CpG thereof) or a “TOGA” nucleotide sequence as they are specific for methyl transferases. In one embodiment, the probe conjugation site comprises a nucleotide sequence specific for a methyl transferase enzyme (e.g. m.Taq1). Thus, the probe conjugation site may comprise the nucleotide sequence TOGA. The methyl transferase enzyme may be an enzyme that uses S-adenosyl methionine (SAM) as a starting reagent. In other words, the methyl transferase enzyme may be an enzyme that binds SAM.

Each of the probe conjugation sites may be separated by at least about 15 base pairs, at least about 30 base pairs, at least about 100 base pairs, or at least about 300 base pairs. The greater the distance between each probe conjugation site, the easier it is to detect a conjugated probe or an analyte bound to a conjugate probe. Most preferably each of the probe conjugation sites is separated by between about 300 base pairs and about 1000 base pairs.

The inventors have surprisingly demonstrated that a modular polynucleotide according to the invention can be created (see Examples 1 and 2). It is a simple and robust solution to investigate a diverse range of analytes (e.g. biomarkers) quickly, simultaneously and at a low cost. Modular polynucleotides can be flexibly designed to carry a range of different probes, or multiple identical probes, depending on the application. Furthermore, the invention provides a novel way of creating modular polynucleotides with improved efficiency, in terms of time, cost and design flexibility.

The skilled person would appreciate that the conjugation of one or more probes to a polynucleotide may be determined by a carrier-enhanced resistive pulse sensing technology (see Loh et al., Anal. Chem. 2018, 90 (23):14063-14071). Thus, the skilled person would also appreciate that modular polynucleotides and (im)mature conjugate subunits according to the invention (or made by methods according to the invention) may be used with a carrier-enhanced resistive pulse sensing technology to detect the binding of analytes in a sample.

According to another aspect of the invention, there is provided a method of creating a recombinant polynucleotide, the method comprising:
(a) (i) using a method according to the invention to create a modular polynucleotide comprising one or more probes each conjugated to a probe conjugation site of a polynucleotide backbone via a UV-sensitive bond, or
(ii) providing a modular polynucleotide comprising one or more probes each conjugated to a probe conjugation site of a polynucleotide backbone via a UV-sensitive bond; and
(b) creating a recombinant polynucleotide by exposing the modular polynucleotide by to UV light in order to cleave the UV-sensitive bond(s) of the modular polynucleotide.

The method according to the invention is advantageous because it would enable the creation of specific sequences that can bridge two or more double-stranded polynucleotides together without the use of a specific restriction site or a large insert. Consequently, less “junk” DNA and more coding DNA can be inserted into viral vectors. Viral vectors have a limited capacity for inserts DNA.

In another aspect, there is provided a method of creating sticky ends on a blunt-ended polynucleotide, the method comprising:
a) (i) conjugating a steric hindering agent to a conjugation site of a polynucleotide comprising a first blunt end and/or a second blunt end, or
(ii) providing a polynucleotide comprising a first blunt end and/or a second blunt end and a steric hindering agent conjugated to a conjugation site of the double-stranded polynucleotide.

The method may optionally further comprise:
b) cleaving the first blunt end and/or the second blunt end of the polynucleotide using a nuclease to create a first sticky end and/or a second sticky end.

The steric hindering agent may be a probe as defined herein. The steric hindering agent impedes the activity of nucleases (e.g. exonucleases) that may be used to cleave short (i.e. less than 100 base pairs) blunt-ended double-stranded polynucleotides (e.g. an immature conjugate subunit). The inventors believe that the conjugation of the steric hindering agent to the conjugation site prevents that exonuclease from continuing its cleavage activity along the entire length of the polynucleotide comprising the sticky end(s). Consequently, the method according to the invention enables the blunt ends of a short double-stranded polynucleotide to be converted into “sticky ends” without converting the entire double-stranded polynucleotide into a single-stranded polynucleotide.

Each probe may be conjugated to a separate polynucleotide using any method known in the art. The conjugating step may comprise chemically-modifying or functionalising the polynucleotide with an adaptor to facilitate conjugation of the probe to the polynucleotide. Thus, the conjugating step may comprise chemically- modifying or functionalising the probe conjugation site (e.g. TCGA or a CpG of a CpG island) of the double-stranded polynucleotide with an adaptor. One or more, two or more, three or more, or four more nucleotides of the probe conjugation site may be chemically modified. Specifically, the conjugating step may comprise chemically-modifying or functionalising the probe conjugation site of the polynucleotide with an azide (N3) group or a peptide adaptor. Preferably the conjugating step comprises chemically attaching an azide group to the probe conjugation site. Preferably the nucleotides of the probe conjugation site are not terminal base-paired nucleotides (i.e. nucleotides located within four nucleotides of the 5′-basepaired terminus or the 3′- basepaired terminus of the polynucleotide). Preferably the probe conjugation site is at least 15 nucleotides from the 5′-terminus and the 3′-terminus of the polynucleotide.

The conjugating step may comprise attaching an azide group to the polynucleotide by contacting the polynucleotide with an azide donor. The azide donor may be a substance comprising an azide group and that has the ability to donate the azide group without reacting to other groups on the recipient molecule. The azide donor may be RAdoHcy-8-Hy -PEG-N3, a modified SAM molecule containing an azide group (rather than a methyl) or an azide modified nucleotide. Preferably the azide donor is RAdoHcy-8-Hy -PEG-N3 or a modified SAM molecule containing an azide group. The conjugating step may comprise chemically-modifying or functionalising the polynucleotide with an azide (-N3) group by contacting the polynucleotide with an azide donor (e.g. RAdoHcy-8-Hy-PEG-N3 or a SAM molecule containing an azide group) in the presence of a catalyst, such as an enzyme. The enzyme may be a methyl transferase. Preferably the methyl transferase is Taql methyl transferase (EC2.1.1.72). The conjugating step may comprise chemically-modifying or functionalising the polynucleotide with an azide (N3) group by contacting the polynucleotide with an azide donor (e.g. RAdoHcy-8-Hy-PEG-N3 or a SAM molecule containing an azide group) in the presence of a methyl transferase, such as Taql methyl transferase (EC2.1.1.72) for about 30 minutes to about 90 minutes at about 50 oC.

The conjugating step may comprise chemically-modifying or functionalising the probe to facilitate conjugation to the polynucleotide without interfering with the ability of the probe to bind an analyte. Thus, the skilled person would appreciate that the probe should be functionalised in a region that is not responsible for binding to an analyte. Thus, in embodiments in which the probe is an antibody, the antibody should not be functionalised in or near the antibody binding region (e.g. Fab). The antibody should be functionalised in a region such as the Fc region. For example, in embodiments where the probe is an antibody, the terminus of Fc region (e.g. CH3 or CH2) of the antibody is functionalised. In embodiments where the probe is a scFv antibody, the linker is functionalised. In embodiments where the probe is a single stranded DNA/RNA, the 5′end of the single stranded DNA/RNA is functionalised with an amine group for conjugation. In embodiments where the probe is a single-stranded polynucleotide, the 3′end is functionalised.

The conjugating step may comprise conjugating the probe directly or indirectly to the polynucleotide. The conjugating step may comprise covalently conjugating the probe directly to the polynucleotide. The conjugating step may comprise conjugating the probe to the polynucleotide via a photocleavable bond (e.g. a UV-sensitive bond).The conjugating step may comprise conjugating (e.g. covalently conjugating) the probe directly or indirectly to the polynucleotide conjugation site. Preferably the probe is not conjugated to a terminal base-paired nucleotide. Preferably the probe conjugation site is not located within 15 base pairs from the 5′-terminus or the 3′-terminus of the polynucleotide. The probe may be conjugated to a polynucleotide that has been chemically-modified or functionalised. The probe may be conjugated to a functional group or chemical group of a nucleotide that has been chemically-modified or functionalised. The conjugating step may comprise conjugating the probe to the double-stranded polynucleotide via a linker (e.g. DBCO-NHS ester, a peptide nucleic acid, a histidine tag). The conjugating step may comprise covalently conjugating the probe to the double-stranded polynucleotide via a linker (e.g. DBCO-NHS ester, a peptide nucleic acid or a histidine tag). Thus, the conjugating step may comprise chemically-modifying or functionalising the probe with a DBCO-NHS ester, a peptide nucleic acid or a histidine tag. Preferably the conjugating step comprises chemically-modifying or functionalising the probe with a DBCO-NHS ester. The conjugating step may comprise chemically-modifying or functionalising a terminal monomer of the probe with a DBCO-NHS ester. The conjugating step may comprise chemically-modifying or functionalising a terminal monomer with an amine group. The conjugating step may comprise chemically-modifying or functionalising a terminal amine group of the probe with a DBCO-NHS ester. The conjugating step may comprise chemically-modifying or functionalising the probe with an amine group, followed by functionalising the amine group with a DBCO-NHS ester. The skilled person would appreciate how to functionalise a variety of different types of probes with an amine group, particularly the terminal monomer of the probe. The conjugating step may comprise chemically-modifying or functionalising the probe by contacting it with a DBCO-NHS ester. The conjugating step may comprise chemically-modifying or functionalising a terminal amine group of the probe by contacting it with a DBCO-NHS ester. The probe or amine group of the probe may be contact with the DBCO-NHS ester at group at about 18° C. to 25° C. for about 2 hours to about 16 hours.

Thus, the conjugating step may comprise contacting a functionalised polynucleotide with a functionalised probe. In one embodiment, the conjugating step comprises contacting a polynucleotide functionalised with a peptide adaptor with a probe functionalised with a PNA or a histidine tag (e.g. a histidine-tagged antibody), in order to conjugate the probe to the polynucleotide. In another embodiment, the conjugating step comprises contacting a polynucleotide functionalised or chemically modified with an azide group with a probe functionalised with a DBCO-NHS ester, in order to conjugate the probe to the polynucleotide. The probe conjugation site of the polynucleotide may be functionalised with an azide group.

The conjugating step may comprise contacting a polynucleotide functionalised with an azide group with a functionalised probe in an azide-alkyne reaction, preferably a copper-free azide-alkyne reaction. The polynucleotide functionalised with an azide group may be contacted with a probe functionalised with a DBCO-NHS ester at about 18° C. to 25° C. for about 1 hour to about 2 hours, or at about 2° C. to about 6° C. for about 16 hours to 24 hours. Preferably the polynucleotide functionalized with an azide group is contacted with a probe functionalised with a DBCO-NHS ester at about 18° C. to 25° C. for about 1.5 hours, or at about 4° C. for about 16 hour to 24 hours.

The conjugating step may comprise conjugating the probe to a probe conjugation site of the double-stranded polynucleotide via a photocleavable bond (e.g. a UV-sensitive bond).

The double-stranded polynucleotide of the (im)mature conjugate subunit may be a polynucleotide comprising artificial nucleotides or natural polynucleotides. One or more of the nucleotides may comprise an epigenetic modification, such as a methylation. The double-stranded polynucleotide may be DNA. Thus, the polynucleotide may be DNA comprising a probe conjugation site. The probe conjugation site may comprise or consist of a nucleotide sequence specific for a transferase enzyme (e.g. the nucleotides TCGA, or a CpG of a CpG island).

The probe conjugation site may be positioned at least 8 base pairs/nucleotides from the 5′-terminal nucleotide/base pair and at least 8 base pairs/nucleotides from the 3′-terminal nucleotide/base pair. The probe conjugation site may be positioned at least about 15 nucleotides from the 5′-terminus and/or the 3′-terminus; at least about 20 nucleotides from the 5′-terminus and/or the 3′-terminus; at least about 25 nucleotides from the 5′-terminus and/or the 3′-terminus; at least about 30 nucleotides from the 5′- terminus and/or the 3′-terminus; at least about 35 nucleotides from the 5′-terminus and/or the 3′-terminus; at least about 40 nucleotides from the 5′-terminus and/or the 3′-terminus; or at least about 45 nucleotides from the 5′-terminus and/or the 3′-terminus. The probe conjugation site sequence may be positioned between about 15 nucleotides and about 48 nucleotides from the 5′-terminus and the 3′-terminus. Preferably the probe conjugation site sequence is positioned about 30 nucleotides from the 5′-terminus and at least 30 nucleotides from the 3′-terminus. The double-stranded polynucleotide may be between about 20 and about 100 base pairs/nucleotides in length or between about 34 and about 100 base pairs/nucleotides in length. Preferably the double-stranded polynucleotide is about 60 base pairs/nucleotides in length.

The double-stranded polynucleotide of the immature conjugate subunit may comprise blunt ends (e.g. a first blunt end and/or a second blunt end). The double-stranded polynucleotide of the mature conjugate subunit may comprise sticky ends (e.g. 5′- sticky ends or 3′-sticky ends).

The probe may be any agent that selectively or specifically binds to an analyte. The probe may bind selectively or specifically to an analyte. Preferably the probe binds specifically to an analyte. Each or two or more of the probes of the modular polynucleotide may be capable of binding to the same analyte or may be identical. Each or two or more of the probes of the modular polynucleotide may be capable of binding to a different analyte.

The probe may be any probe known in the art. The probe may be a polymer (e.g. a ssRNA, a morpholino or a peptide nucleic acid). There are different classes of probe known in the art, such as polypeptides, proteins and polynucleotides. Thus, the probe may be one or more members selected from the group comprising a polypeptide, a protein (e.g. an antibody or an affimer), a polynucleotide (e.g. DNA or single-stranded DNA), a nanoparticle and an aptamer. Preferably, the probe(s) is/are one or more selected from the group consisting of a single-stranded nucleotide, an antibody, a (functional) fragment of an antibody and an aptamer.

The probe may be DNA, preferably single-stranded DNA, or RNA, preferably single-stranded DNA.

The probe may be an antibody or a (functional) fragment thereof (e.g. a scFv, a VL, a VH, a Fd; an Fv, an Fab, a Fab′, a F(ab′)2, an Fc fragment, or a bispecific antibody) that binds to an analyte. For example, when the invention is being used to detect sepsis biomarkers in a sample, the probe may be an antibody that binds to the sepsis biomarkers Interleukin 6 (IL-6) or Procalcitonin.

The term “antigen-binding region” can mean a region of the antibody having specific binding affinity for its target antigen/analyte. The binding region may be a hypervariable CDR or a functional portion thereof. The term “functional portion” of a CDR can mean a sequence within the CDR which shows specific affinity for the target analyte.

The term “(functional) fragment” of an antibody can mean a portion of the antibody which retains a functional activity. A functional activity can be, for example antigen binding activity or specificity.

The term “VL fragment” can mean a fragment of the light chain of a human monoclonal antibody which includes all or part of the light chain variable region, including the CDRs. A VL fragment can further include light chain constant region sequences.

The term “VH fragment” (nanobody) can means a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-i. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about no amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain. The antibody fragment may alternatively comprise a Fab′2 fragment comprising the hinge portion of an antibody.

The term “F(ab) fragment” can mean a bivalent antigen-binding fragment of a human monoclonal antibody. An F(ab) fragment includes, for example, all or part of the variable regions of two heavy chains-and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR ora functional portion thereof will vary depending on the sequence and size of the CDR. Those skilled in the art can routinely determine which residues comprise a particular CDR given the variable region amino acid sequence of the antibody.

The mature conjugate subunit may be created by cleaving the double-stranded polynucleotide of the immature conjugate subunit with an enzyme. The enzyme may be a nuclease enzyme. Most preferably the enzyme is an exonuclease. Exonucleases are enzymes that cleave terminal 3′ and/or 5′ nucleotides from a polynucleotide, such as a blunt-ended polynucleotide, to create sticky ends. The enzyme may be a 5′ exonuclease or a 3′ exonuclease. Preferably the enzyme is a 5′ exonuclease, such as a T5 exonuclease.

A sticky end refers to a series of unpaired nucleotides in a double stranded oligonucleotide. Sticky ends are created by nucleases (e.g. endonucleases or exonucleases) creating a staggered cut in a double-stranded polynucleotide. Nucleases typically have recognition sequences that vary in the number of nucleotides from 4 to 6 and even 8. The longer the sticky ends, the higher the melting temperature of the double-stranded polynucleotide and the higher the number of potential sequences for recognition. Also, the longer the sticky ends, the greater the possibility of a single-strand break (DNA base hydrolysis). Thus, the sticky ends of the nucleotides referred to herein may be at least about 5, at least or about 6 or at least or about 7 nucleotides in length. Preferably the sticky ends of the nucleotides are about 15 to about 25 nucleotides in length. Most preferably the sticky ends of the nucleotide are about 20 nucleotides in length. Sticky ends are preferably 20 nucleotides in length because it provides 160,000 potential sequences for recognition. Sticky ends that are about 20 nucleotides in length are also preferred because they provide a moderately high melting temperature, thus preventing separation of the strands during enzymatic construction of the modular polynucleotide.

The length of the sticky ends is dependent on the nuclease used to cleave the polynucleotide. The nuclease may be an exonuclease, preferably a T5 exonuclease. T5 exonucleases create sticky ends that are about 20 nucleotides in length.

The skilled person will appreciate that the enzyme used to create sticky ends in a double-stranded polynucleotide can also be used to cleave a separate double-stranded polynucleotide so as to make the sticky ends of both polynucleotides complementary. Thus, for example, the double-stranded polynucleotide of an immature conjugate subunit and a double-stranded polynucleotide spacer may be cleaved by th same nuclease (such as an exonuclease, e.g. T5 exonuclease) so as to create complementary sticky ends that may be used to anneal the spacer and subunit together to form a modular polynucleotide. Consequently, the modular polynucleotide may be created by one-pot synthesis (mixing all of the reactants in a single reaction vessel as opposed to making the modular polynucleotide in a stepwise fashion). The skilled person will also appreciate that one-pot synthesis can be used to create a modular polynucleotide according to the invention due to the polynucleotide of each immature conjugate subunit comprising a nucleotide sequence that once cleaved will be complementary to a separate mature conjugate subunit. Thus, once each polynucleotide (i.e. immature conjugate subunit and double-stranded polynucleotide spacer) in the one-pot synthesis reaction has been cleaved by a single nuclease (e.g. T5 exonuclease), only the polynucleotides with complementary sticky ends will anneal together. Thus, the order of the probes in a modular polynucleotide may be arranged at user's discretion due to the modular nature of the mature conjugate subunits. The sticky ends referred to herein may be 5′ sticky ends or 3′ sticky ends.

The spacer may be a double-stranded polynucleotide. The spacer referred to herein may be a double-stranded polynucleotide comprising a nucleotide sequence that once cleaved (e.g. with an exonuclease, such as T5 exonuclease) will comprise one or two sticky ends that are complementary to the sticky ends of a mature conjugate subunit or will be complementary to the sticky ends of a mature conjugate subunit once it has been formed.

The spacer may comprise artificial nucleotides or natural polynucleotides. In one embodiment the double-stranded polynucleotide is DNA. One or more of the nucleotides may comprise an epigenetic modification, such as a methylation.

The double-stranded polynucleotide spacer (e.g. DNA) may be at least about 100 or more, 200 or more, 300 or more, 500 or more, 1000 or more base pairs in length. The double-stranded polynucleotide spacer (e.g. DNA) may be between about 300 and 2000 base pairs in length or between about 500 base pairs and about 1000 base pairs in length.

The presence of the spacer within the modular polynucleotide enables the distance between each probe to be controlled so as to increase the resolution of each signal produced by each probe but also to prevent each probe from being too spaced apart. Furthermore, the spacer can be used to control the order of the probes in the modular polynucleotide according to the invention.

The annealing step may comprise using any method known in the art to anneal complementary sticky ends together. The annealing step may comprise contacting a first mature conjugate subunit with a second or several other mature conjugate subunits. The annealing step may comprise contacting a mature conjugate subunit with a double-stranded polynucleotide spacer. The annealing step may comprise an enzyme. Preferably the annealing step occurs in the presence of an enzyme. The enzyme may be a DNA ligase and/or a DNA polymerase. DNA ligase joins or catalyses the joining of complementary sticky ends together. DNA polymerase inserts nucleotides into gaps of an incompletely synthesised/annealed double-stranded polynucleotide, such as modular polynucleotide referred to herein.

Preferably contacting a mature conjugate subunit with a double-stranded polynucleotide spacer occurs in the presence of a DNA ligase and a DNA polymerase.

The DNA ligase may be Taq ligase. The DNA polymerase may be Taq DNA polymerase. Contacting a mature conjugate subunit with a double-stranded polynucleotide spacer may occur in the presence of a DNA ligase and a DNA polymerase for about 1 hour to 18 hours at about 40° C. to about 50° C. Performing the annealing step under these conditions ensures that only complementary sticky ends anneal to each other (i.e. prevents mismatching of sticky ends). Mismatched base pairs are unstable at temperatures between about 40° C. to about 50° C.

The modular polynucleotide may comprise two or more different probes that are capable of binding to the same analyte or a different analyte. The modular polynucleotide may comprise two or more different classes of probe that are capable of binding to the same analyte or a different analyte. The modular polynucleotide may comprise two or more identical probes.

The modular polynucleotides and mature conjugate subunits of the invention may be used for a variety of purposes, including for example, disease diagnosis, the food industry (e.g. testing food and water quality), waste analysis (e.g. nuclear and industrial waste analysis), environmental analysis (e.g. soil and atmosphere aspirational analysis).

The inventors have developed a kit comprising components that can be used with a carrier-enhanced resistive pulse technology (e.g. nanopore-based resistive pulse sensing or nanopipette-based resistive pulse sensing technology) to determine if one or more analytes is present in a sample.

Thus, according to a ninth aspect there is provided a kit for determining if one or more analyte(s) is/are present in a test sample, the kit comprising:
a double-stranded polynucleotide spacer; and
an (im)mature conjugate subunit according to the invention or an (im)mature conjugate subunit made by the method according to the invention; or
a modular polynucleotide according to the invention or a modular polynucleotide made by a method according to the invention.

In one embodiment, there is provided a kit for diagnosing a test subject suffering from a medical condition, the kit comprising:
a double-stranded polynucleotide spacer; and
an (im)mature conjugate subunit according to the invention or an (im)mature conjugate subunit made by the method according to the invention; or
a modular polynucleotide according to the invention or a modular polynucleotide made by a method according to the invention,
   wherein the presence of one or more analyte(s) in a bodily sample from a test subject is indicative that the subject suffers from the medical condition, or wherein the absence of the one or more analyte(s) from a bodily sample from a test subject is indicative that the subject suffers from the medical condition.

A kit according to the ninth aspect may be used to make a modular polynucleotide according to the invention. A modular polynucleotide according to the invention may be used to perform multiplex, high-throughput analysis using a carrier-enhanced resistive pulse sensing technology for the detection of different analytes using different probes.

Preferably, the kit further comprises at least one control or reference sample. The kit may comprise a control (e.g. a negative control and/or a positive control). The negative control may be a sample that does not comprise one or more of the analyte(s) to be detected. The positive control may be a sample that comprises one or more of the analyte(s) to be detected. The kit may comprise a buffer for the samples. The kit may comprise one or more enzymes selected from the group consisting of a DNA polymerase, a nuclease (e.g. exonuclease) and a DNA ligase.

The tenth aspect provides a method of determining if one or more analyte(s) is/are present in a test sample, the method comprising:
i. contacting a modular polynucleotide according to the invention or a modular polynucleotide made by a method according to the invention with a test sample; and then
ii. analysing the modular polynucleotide using a carrier enhanced-resistance pulse sensing technology to determine if one or more analyte(s) is/are present in the test sample.

The test sample may be an environmental sample (e.g. a soil sample or atmospheric sample), a food industry, a water sample, a waste sample (e.g. nuclear or industrial waste sample) or a bodily sample that has been taken from a test subject. Thus, the method according to the invention may not comprise taking a sample from a test subject or performing surgery on a test subject.

The method according to the tenth aspect may be used to diagnose if a test subject suffers from a medical condition or disease.

Thus, the eleventh aspect provides a method of diagnosing a test subject with a medical condition, the method comprising:
i. contacting a modular polynucleotide according to the invention or a modular polynucleotide made by a method according to the invention with a bodily sample taken from the test subject; and then
ii. analysing the modular polynucleotide using a carrier enhanced resistance pulse sensing technology to determine if one or more analyte(s) is/are present in the bodily sample, wherein the presence in the sample of the one or more analyte(s) is/are indicative that the subject has the medical condition, or wherein the absence from the sample of the one or more analyte(s) is/are indicative that the subject has the medical condition.

The method according to the eleventh aspect is advantageous because it can be used to detect several different types of biomarkers simultaneously. Thus, the method provides a faster way to detect several different analytes and can be used to provide a more accurate diagnosis than known methods.

The method of the eleventh aspect may comprise administering a therapeutic agent that treats the medical condition or disease to a subject.

Therefore, in a twelfth aspect, there is provided a method of treating a subject suffering from a medical condition, the method comprising:
diagnosing a test subject with a medical condition using a method according to the invention; and
administering a therapeutically effective amount of a therapeutic agent for treating the medical condition.

In another aspect, there is provided a therapeutic agent for use in treating a medical condition in a subject diagnosed with a medical condition using a method according to the invention.

In an thirteenth aspect, there is provided a method of determining the efficacy of a therapeutic agent being used to treat a subject's medical condition, the method comprising:
i. diagnosing a test subject with a medical condition using a method according to the invention;
ii. administering a therapeutically effective amount of a therapeutic agent for treating the medical condition;
iii. contacting a modular polynucleotide- according to the invention or a modular polynucleotide made by a method according to the invention with a test sample; and
iv. analysing the modular polynucleotide using a carrier-enhanced resistance pulse sensing technology to determine if one or more analyte(s) is/are present in the test sample, wherein the presence or absence of the one or more analyte(s) in the test sample is indicative of the efficacy of the therapeutic agent being used to treat the test subject's medical condition.

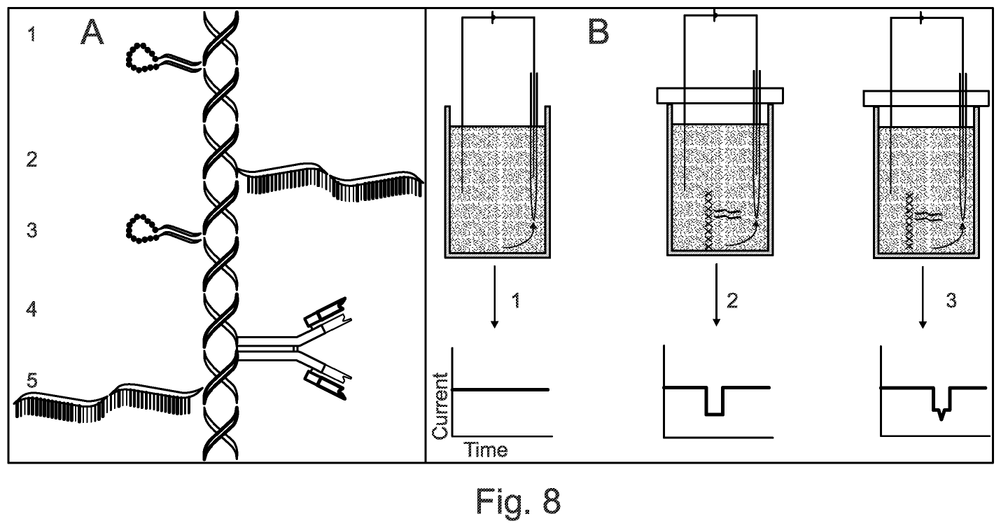

Resistive pulse sensing technology requires two solutions to be separated by a narrow channel. A voltage is applied across the channel and (charged) molecules from one solution will move through the channel in the direction of the electric field. As they cross through the channel, the current passing between the electrodes will change. The change in current, and the duration of the change are directionally proportional to the widest diameter, and dimensions of the molecule passing through (see FIG. 8). Examples of carrier-enhanced resistive pulse sensing technology include nanopore-based resistive pulse sensing, such as a biological nanopore (e.g. a Phi29 Connector channel), and nanopipette-based resistive pulse sensing technology.

The analyte is an agent that is capable of being bound by a probe. The analyte may be any substance present in a test sample (e.g. a biological or bodily sample or a non- biological sample or an environmental sample, a waste sample, a food sample or a water sample). Thus, the analyte may be a biological agent or a chemical agent.

The sample may be a fluid, such as a liquid or a gas. The sample may be a gas that has been condensed into a liquid. Preferably the sample is a liquid. Preferably the analyte is at least 2 nm in length.

The sample may be a biological sample, such as a biological liquid. Thus, the analyte can be an analyte that is secreted from cells. Alternatively, the analyte can be an analyte that is present inside cells such that the analyte must be extracted from the cells before the invention can be carried out. Alternatively, the analyte can be an analyte that is present in a sample of fluid in which the biological organism is located.

Thus, the sample may be in vitro, in vivo or ex vivo. Thus, the invention may be carried out in vitro on a sample obtained from or extracted from a biological organism.

The biological organism or test subject may be a bacterium, a protista, a fungi, a plant or an animal. The biological organism may be a mammal, such as a human. Thus, the sample may be a bodily sample, such as a mammalian bodily sample, e.g. a human bodily sample. The sample typically comprises a biological fluid sample of the organism (e.g. a human). The biological fluid sample may be cerebrospinal fluid (CSF), urine, lymph, saliva, mucus or amniotic fluid.

The biological organism may be a commercially farmed animal, such as a fish, a horse, cattle, sheep or a pig; a pet, such as a cat or a dog; or a lab animal such as a mouse, a rat, a hamster or a guinea pig. The plant may be a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa or cotton. The plant may be a tree, such as Hymenoscyphus fraxineus.

The analyte may be an amino acid, a polypeptide, a carbohydrate (e.g. a polysaccharide or a disaccharide or a monosaccharide), lipids (fat), vitamin, a mineral, a metabolite or a polynucleotide. The polypeptide may be a protein. The protein can be an enzyme, antibody, complement protein (immune reaction), hormone, growth factor or growth regulatory protein, such as a cytokine, a structural protein (such as actin), a cellular receptor proteins (MHC), a transporter protein, glycosylated proteins.

The protein may be a bacterial protein, a fungal protein, a viral protein, a plant protein, an animal protein, a protista protein or a parasite-derived protein.

The analyte may be a biomarker. The biomarker may be any biomarker known in the art that is capable of being bound by a probe. For example, the analyte may be interleukin-6, which is a marker of sepsis; procalcitoninin, which is a marker of a bacterial infection etc.

The sample may be a non-biological sample or a chemical sample. The non-biological sample is preferably a fluid sample, e.g. a liquid sample or gaseous sample. Examples of a non-biological sample include surgical fluids, water such as drinking water, sea water or river water, and reagents for laboratory tests.

Thus, the invention may be carried out on a sample that is known to contain or suspected to contain the analyte. The invention may be carried out on a sample that contains one or more analytes whose identity is unknown. Alternatively, the invention may be carried out on a sample to confirm the identity and/or concentration of one or more analytes whose presence in the sample is known or expected.

According to another aspect of the invention, there is provided a method of tagging a polynucleotide with an azide group, the method comprising: contacting a polynucleotide comprising a TOGA target site with an azide donor in order to tag the target site of the polynucleotide with an azide group.

The polynucleotide may be a double-stranded polynucleotide referred to herein, or a single-stranded polynucleotide.

An azide donor is a substance comprising an azide group with the ability to freely donate the azide group without reacting to other groups on the recipient molecule. Thus, the azide donor may be RAdoHcy-8-Hy-PEG-N3, a modified SAM molecule containing an azide group or an azide modified nucleotide.

The contacting step may be performed in the presence of a catalyst, such as an enzyme. The enzyme may be a methyl transferase, preferably Taql methyl transferase (EC 2.1.1.72)

The contacting step may be performed in the presence of a methyl transferase for at least about 20 minutes, at least about 30 minutes, at least about 30 minutes. The contacting step may be performed in the presence of a methyl transferase (e.g.Taql methyl transferase) for between about 20 minutes and 24 hours. Preferably the contacting step is performed in the presence of a methyl transferase for between about 30 minutes and 2 hours, or between about minutes and 90 minutes.

The contacting step may be performed in the presence of a methyl transferase (e.g. Taql methyl transferase) at about 34° C. to about 55° C., preferably at about 45° C. to about 55° C. Most preferably the contacting step is performed in the presence of a methyl transferase for about minutes to about 90 minutes at about 50° C.

The term “one or more” can mean two or more, three or more, four or more, five or more, six more, seven or more, eight or more, nine or more, 10 or more, 15 or more, or 20 or more, 50 or more, 100 or more, 200 or more, 300 or more, 500 or more or 1000. The term “one or more” can alternatively mean “all”.

A conjugation site or a probe conjugation site may comprise “a nucleotide sequence specific for a transferase enzyme”.

“A nucleotide sequence specific for a transferase enzyme” can refer to a nucleotide sequence that comprises nucleotides that can be (specifically) modified by a transferase enzyme, e.g. a CpG island or a CpG thereof.

The term “CpG island” may refer to a region of DNA that comprises a large number of CpG dinucleotide repeats. The region of DNA may be at least 200 nucleotides in length and have a CpG% of at least 50%.

The term a “(functional) fragment” thereof can refer to an analyte-binding fragment.

As would be clear to the skilled person, the meaning of the term “probe” as used herein depends on the context it is used in. For example, in the context of a system for diagnosing an oral disease in a subject, the probe is a device configured to collect a fluid sample from an oral cavity of the subject. In another example, in the context of a method of making an immature conjugate subunit, the probe may be any agent that selectively or specifically binds to an analyte.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Embodiments of the invention will now be described by way of example and with reference to the accompanying figures, in which:

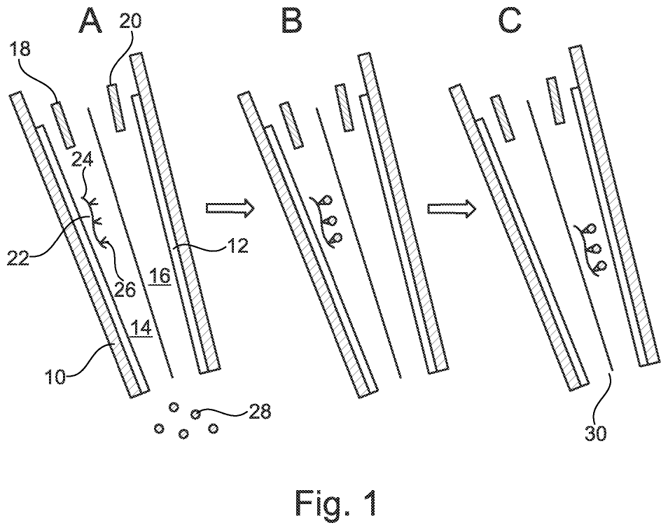

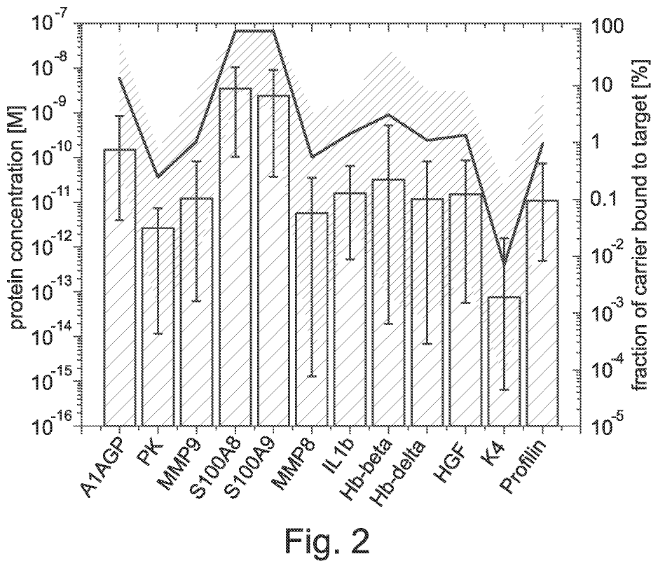

FIG. 1 is a schematic drawing showing the process of detecting target analytes in a fluid sample obtain from the oral cavity of a subject, in accordance with an embodiment of the invention; and

FIG. 2 shows simulation results based equilibrium binding theory with one target analyte binding site per carrier molecule (equilibrium constant KD=10⁻¹⁰ M (typical value for antibody/antigen interactions), c(carrier)=10⁻⁹ M), for upper, mean and lower concentration values of 12 identified marker proteins. Left axis: bar chart, concentration values in saliva. Right axis: fraction of capture probe bound to target, for determining the target concentration.

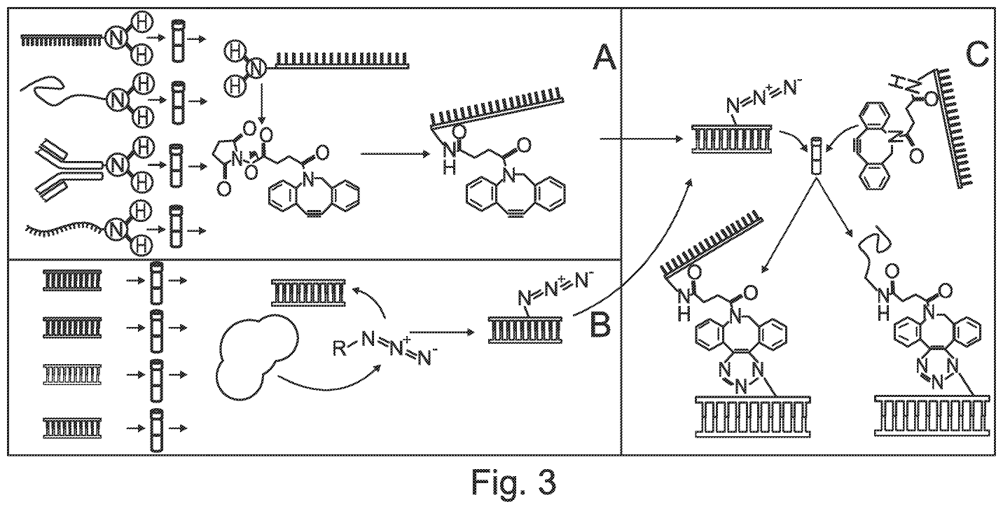

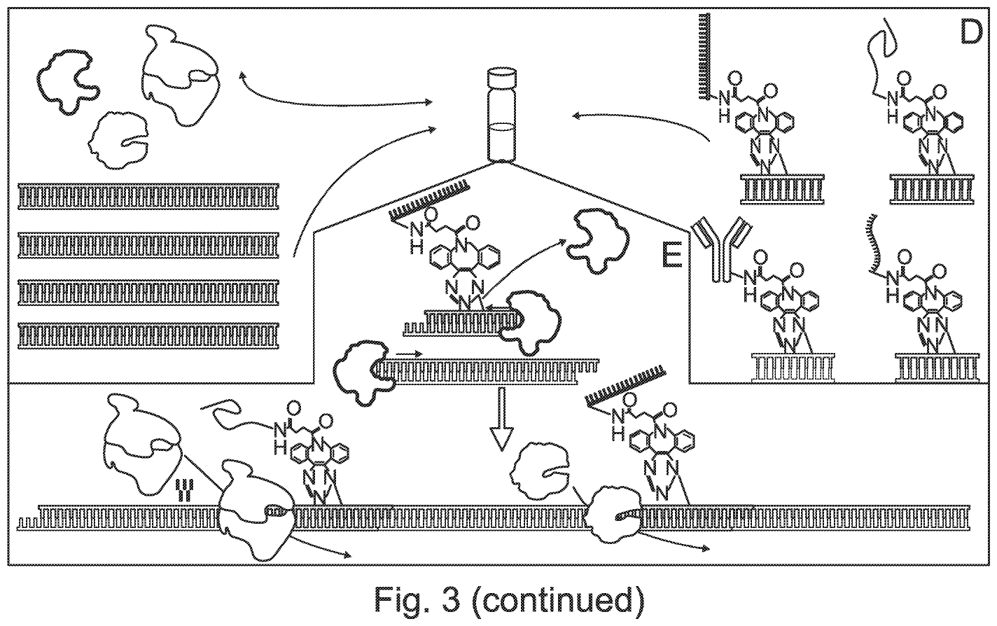

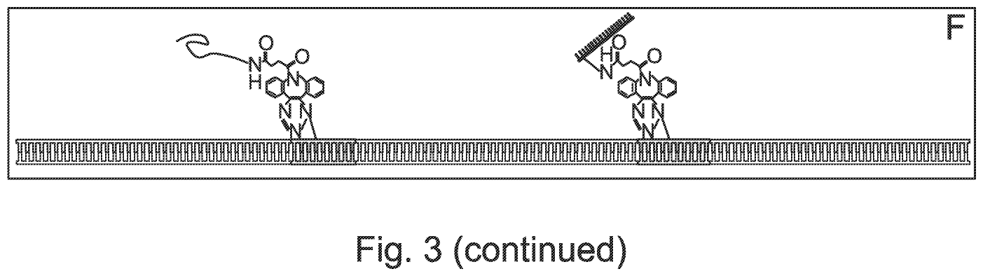

FIG. 3 is a schematic overview of a method of making a modular double-stranded polynucleotide according to the invention (the method is also referred to herein as Sterically Controlled Nuclease Enhanced DNA Assembly (SCoNE DNA Assembly, Formally IADL));

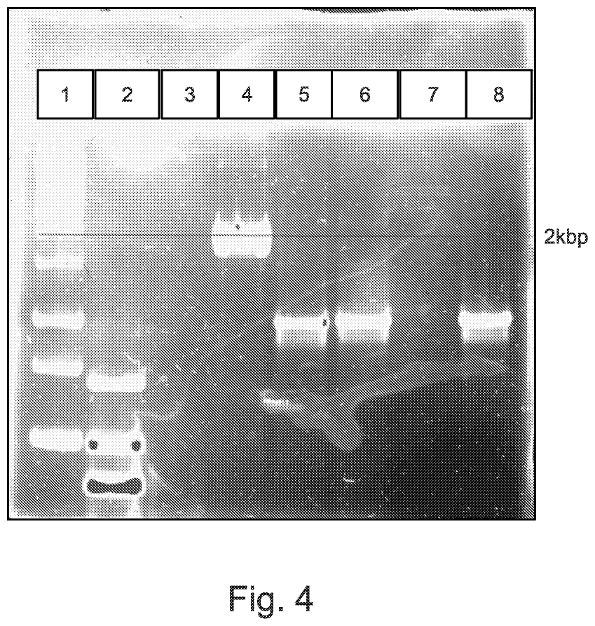

FIG. 4 is a 1% agarose gel run at 75V for 45 minutes showing an N of 1 SCoNE. (1) Gene Ruler 1 kbp, (2) Gene Ruler low range, (3) 200 bp DNA fragment (NoLimit, Thermo Scientific™), (4) 2 kbp DNA fragment (NoLimit, Thermo Scientific™), (5) Negative control, (6) IADL 1 pM, (7) Positive assembly fragment, and (8) IADL 5 pM;

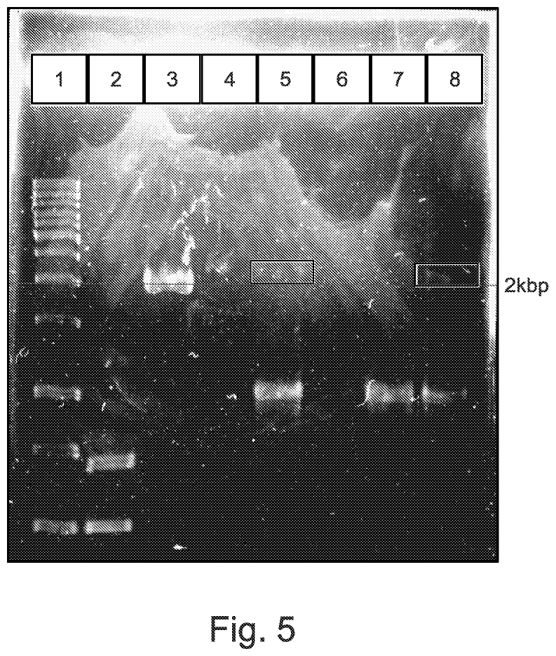

FIG. 5 is a 1% agarose gel run at 75V for 45 minutes showing an N of 2 SCoNE experiment. (1) Gene Ruler 1 kb, (2) Gene Ruler low range, (3) 2 kbp DNA fragment (NoLimit, Thermo Scientific™), (4) Short P1 strand (self-assembly check), (5) IADL (SCoNE) 1 pM, (6) Positive assembly fragment, (7) Negative control, and (8) IADL (SCoNE) 5 pM;

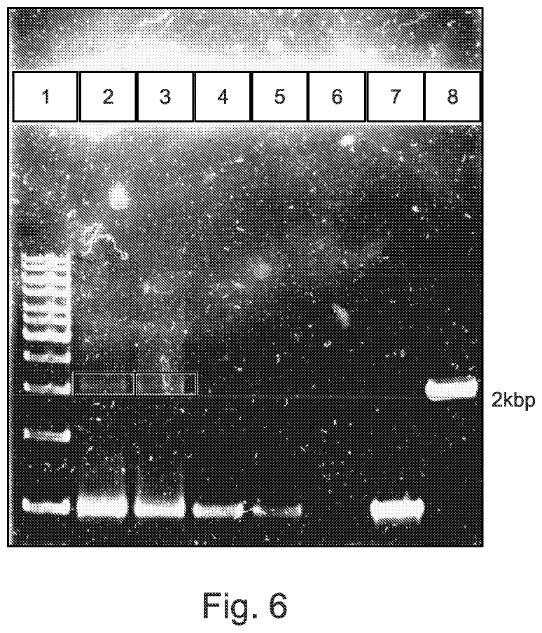

FIG. 6 is an agarose gel run at 75V for 60 minutes showing an N of 3 and 4 SCoNE experiment. (1) Gene Ruler 1 kbp, (2) IADL (SCoNE) 1pM, (3) IADL (SCoNE) 0.5 pM, (4) IADL (SCoNE) 0.25 pM, (5) IADL (SCoNE) 0.125 pM, (6) Positive Control, (7) Negative control, and (8) 2 kbp fragment (NoLimit, Thermo Scientific™);

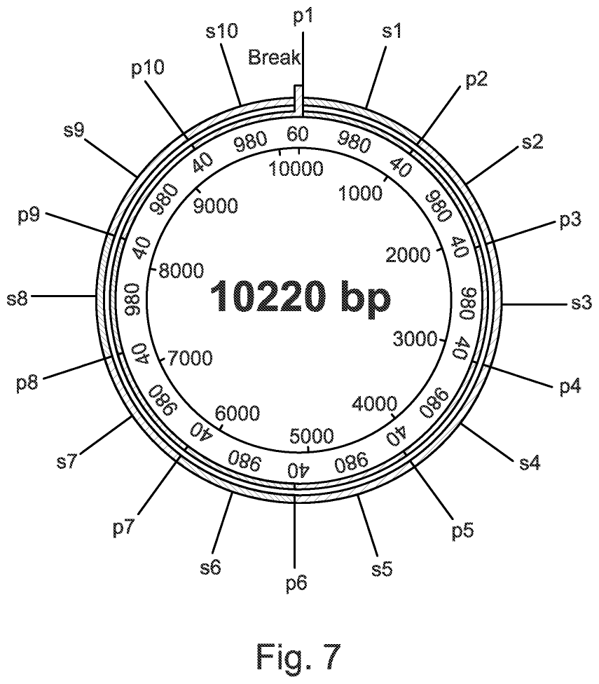

FIG. 7 shows a Gibson fragment assembly highlighting the probe attached fragments (p) and the spacer fragments (s) of dsDNA. It also illustrates that the strand will not circularise due to the break formation between probe fragment 1 and spacer fragment 10; and

FIG. 8 is (A) an illustration of the DNA double helix backbone with adjoining capture probes for metabolites (probes A1 and A3 are aptamers; probes A2 and A5 are single-stranded DNA or RNA; and probe A4 is an antibody that binds a protein). (B) The experimental setup highlighting the differences in the signal obtained using a carrier-enhanced resistive pulse sensing technology when: (B1) no probe is bound, (B2) the analyte is not bound to the probe, and (B3) the analyte is bound to the probe.

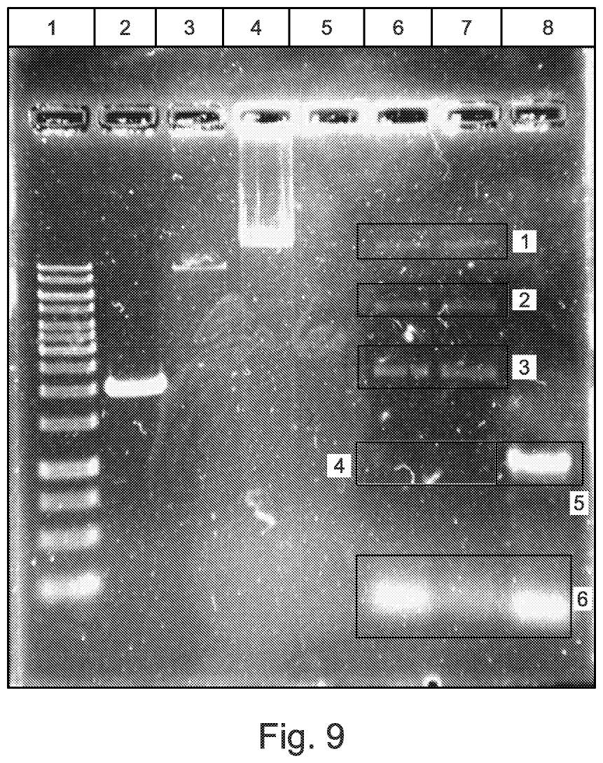

FIG. 9 is a 1% agarose gel highlighting the ability to create decamer SCoNE structures (lanes 6 and 7). Lane 1 Gene Ruler 1 kbp (Thermo Scientific™), lane 2 2 kbp fragment (NoLimit, Thermo Scientific™), lane 3 10 kbp fragment (NoLimit, Thermo Scientific™), lane 4 whole λ DNA (Thermo Scientific™), lane 5 PCR amplified construct, lane 6 decamer SCoNE 1 pM (N=1), lane 7 decamer SCoNE 1 pM (N=2), lane 8 negative control. We show our ability to generate decamer structures (lanes 6 and 7, 18.1).

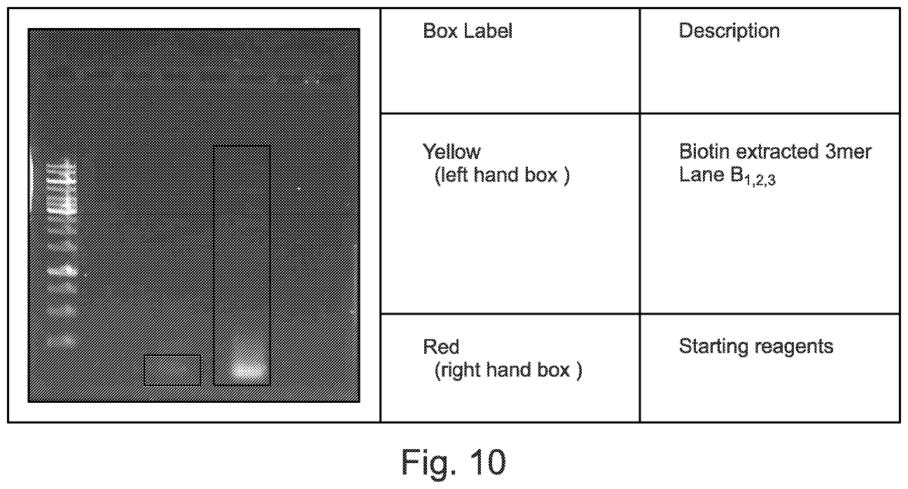

FIG. 10 shows a 1% agarose gel run at 75V for 45 minutes which shows that the inventors were are able to extract biotin labelled p strands (yellow box, left hand box) from the reaction mixture (red box, right hand box) with lane 1, gene ruler, lane 4 biotin extracted p strands, lane 6 reaction mixture prior to assembly.

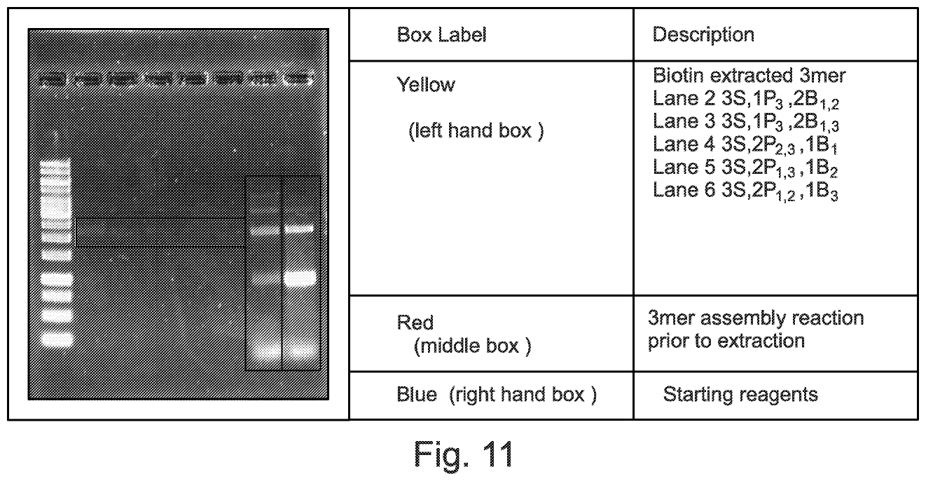

FIG. 11 shows a 1% agarose gel run at 75V for 45 minutes illustrating the inventors ability to assemble, and extract 3mer scone structures (yellow box, left hand box) from an assembled sample (red box, middle box). This also shows the need for an extraction method comparing to the starting reaction mixture (blue box, right hand box).

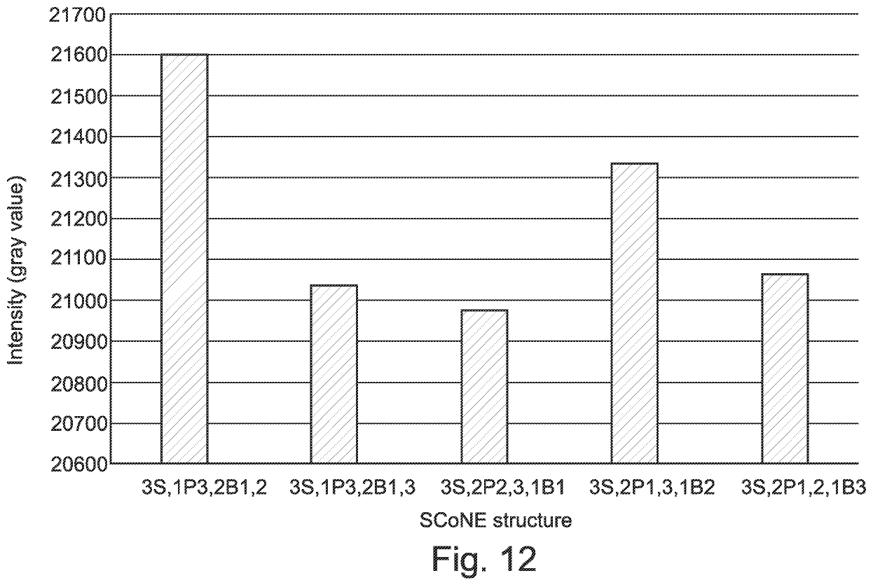

FIG. 12 show respective intensity compared to biotin group positioning.

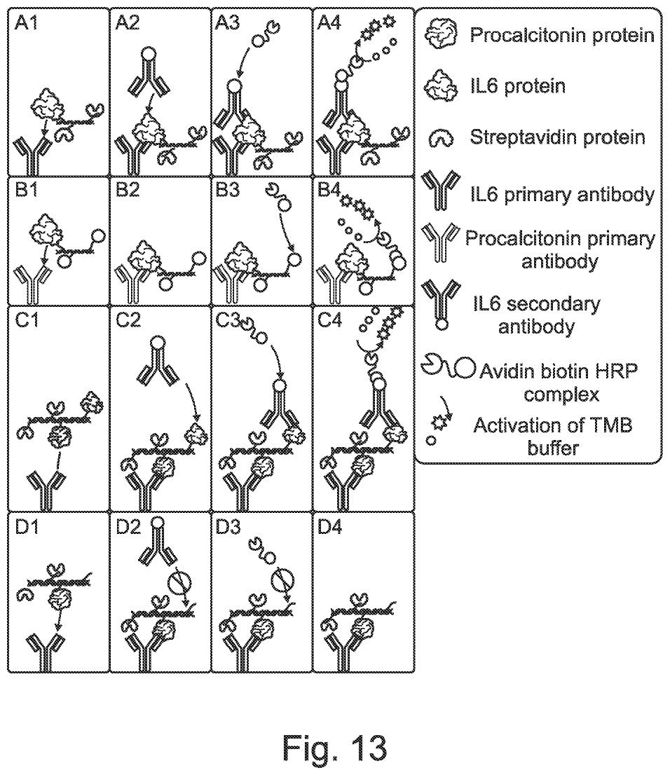

FIG. 13 shows ELISA and newly adapted ELISA protocols. Panel A shows a normal ELISA using the 3mer SCoNE structure for protein isolation using streptavidin to bind reactive biotin groups. A1 shows SCoNE bound protein binding to primary IL6 antibody. A2 shows Secondary IL6 antibody binding to protein. A3 shows ABC binding to biotin labelled antibody. A4 shows ABC converting TMB buffer to coloured compound for measurement. Panel B shows an ELISA using the 3mer SCoNE structure, but removing the secondary antibody and measuring the protein concentration based on the biotin binding alone. B1 shows SCoNE bound protein binding to primary IL6 antibody. B2 shows Secondary IL6 antibody not added. B3 shows ABC binding to biotin labelled SCoNE. B4 shows ABC converting TMB buffer to coloured compound for measurement. Panel C shows a modified ELISA using the 4mer SCoNE structure where the primary antibody binds procalcitonin and the secondary binds IL6. C1 shows SCoNE bound protein binding to primary procalcitonin antibody. C2 shows Secondary IL6 antibody binding to IL6 protein. C3 shows ABC binding to biotin labelled IL6 antibody. C4 shows ABC converting TMB buffer to coloured compound for measurement. Panel D shows a similar experiment to C using the 4mer SCoNE structure where the structure has not been incubated with IL6, therefore the secondary antibody cannot bind and no signal should be observable. D1 shows SCoNE bound protein binding to primary procalcitonin antibody. D2 shows Secondary IL6 antibody added but cannot bind anything so is washed off. D3 shows ABC cannot bind as biotin sites are blocked by streptavidin. D4 shows ABC not present so cannot convert TMB to colour.

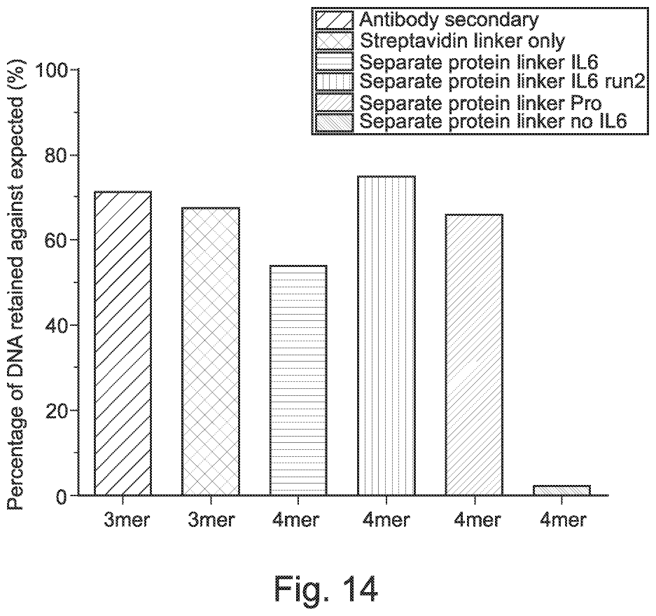

FIG. 14 shows percentage of SCoNE structures retained against expected during ELISA analysis. “Antibody secondary” as referred to in the key of FIG. 14 corresponds to the left most bar of the graph. “Streptavidin linker only” as referred to in the key of FIG. 14 corresponds to the second bar from the left of the graph. “Separate protein linker IL6” as referred to in the key of FIG. 14 corresponds to the third bar from the left of the graph. “Separate protein linker IL6 run 2” as referred to in the key of FIG. 14 corresponds to the fourth bar from the left of the graph. “Separate protein linker Pro” as referred to in the key of FIG. 14 corresponds to the fifth bar from the left of the graph. “Separate protein linker no IL6” as referred to in the key of FIG. 14 corresponds to the sixth bar from the left of the graph.

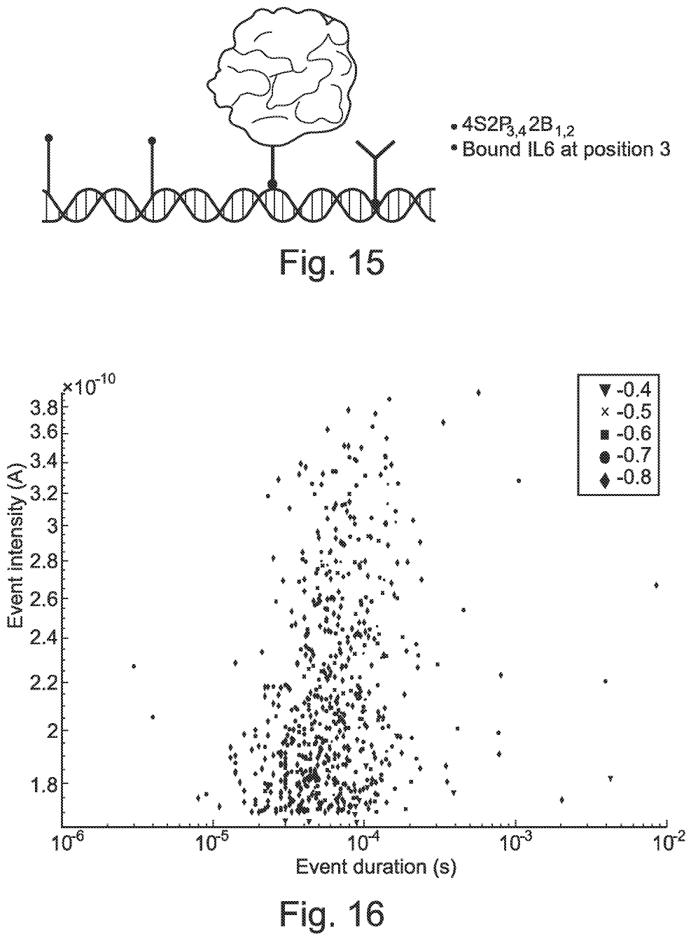

FIG. 15 shows structure of the 4mer with biotin at positions 1 and 2, bound human IL6 on an aptamer at position 3 and a blank Procalcitonin at position 4.

FIG. 16 shows all 4mer translocation events at different biases (V).

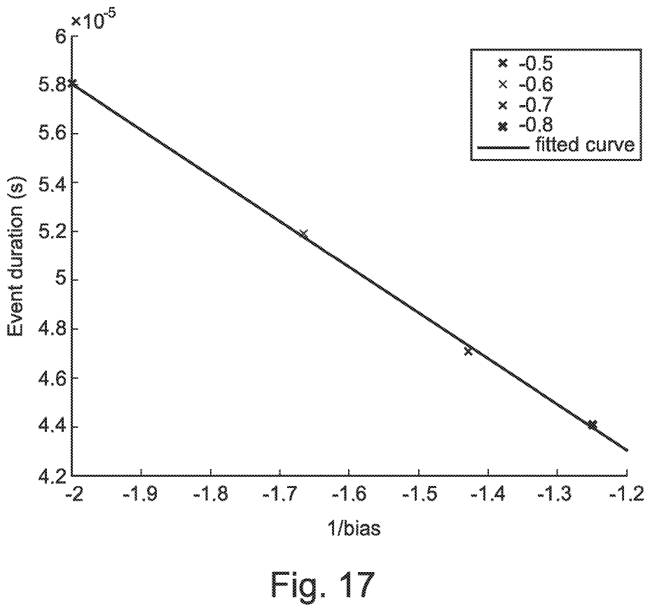

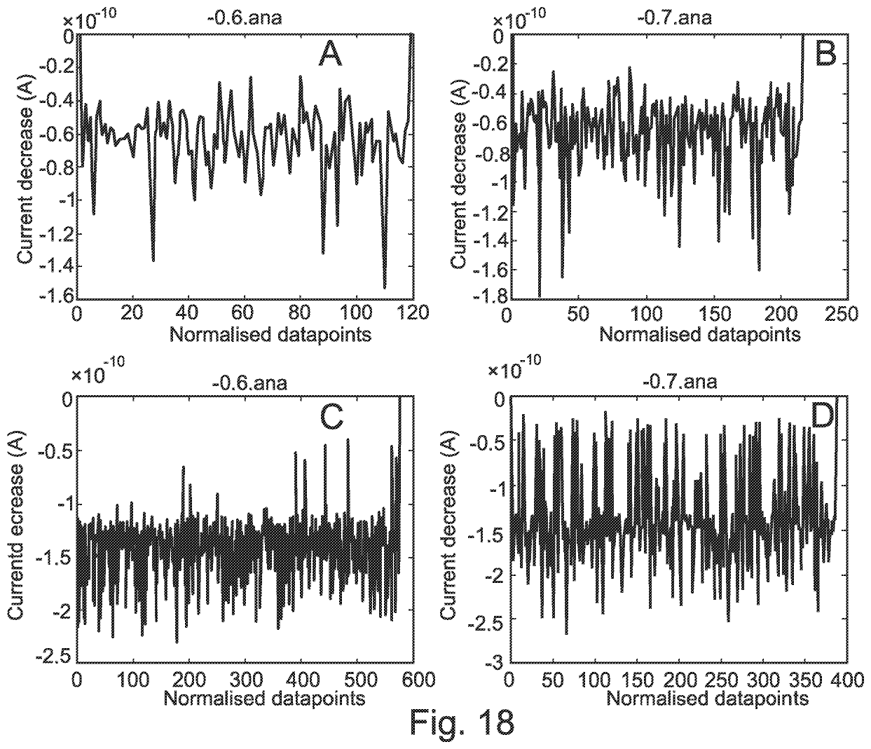

FIG. 17 shows an inverse relationship between bias and event duration. “−0.5” as referred to in the key of FIG. 17 corresponds to the left most data point on the graph, indicated by “x”. “−0.6” as referred to in the key of FIG. 17 corresponds to the second data point from the left on the graph, indicated by “x”. “−0.7” as referred to in the key of FIG. 17 corresponds to the third data point from the left on the graph, indicated by “x”. “−0.8” as referred to in the key of FIG. 17 corresponds to the fourth data point from the left on the graph, indicated by “x”. The values referred to in the key of FIG. 17 correspond to the bias voltage applied. Accordingly, reference to “−0.5”, as referred to in the key of FIG. 17, refers to “−0.5V”. As would be clear to the skillled person, reference to “−0.6” in the key of FIG. 17 thus refers to “−0.6V” and so on.

FIG. 18 shows a comparison between SCoNE DNA average events at −0.6 and −0.7V (A and B) to bare DNA at the same biases (C and D).

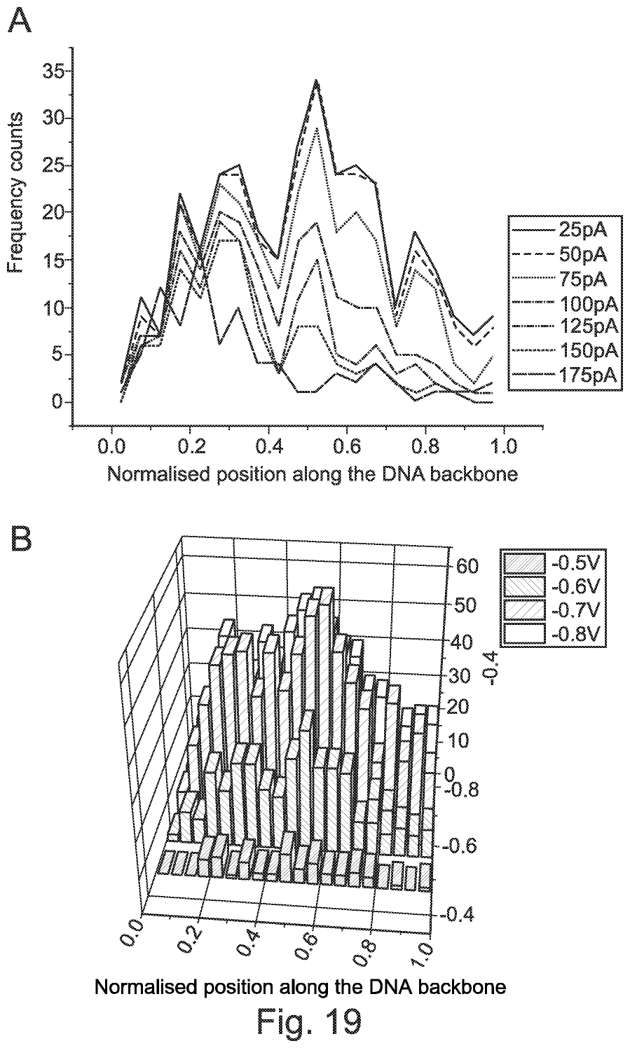

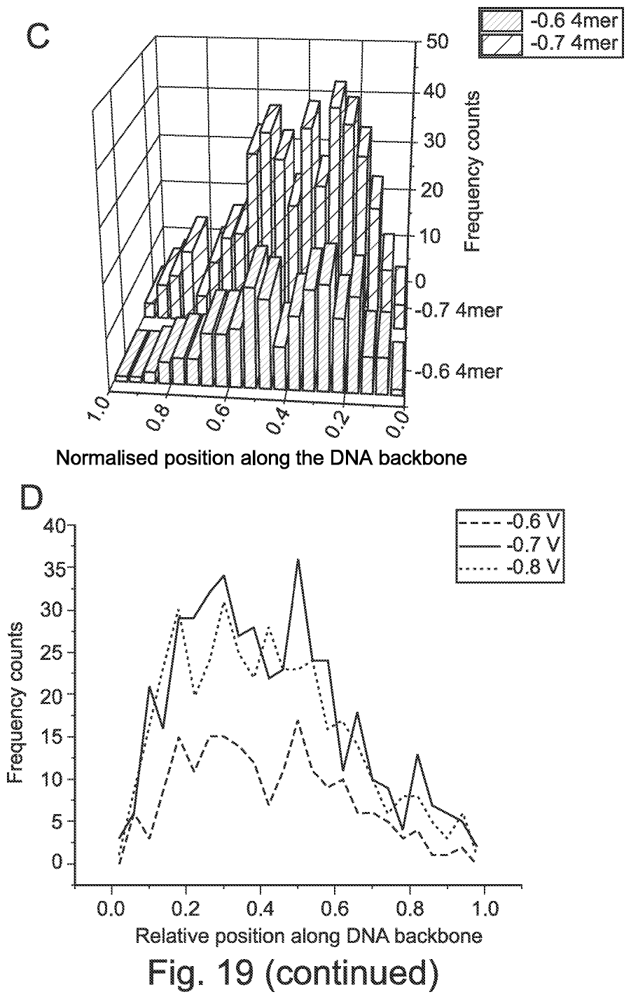

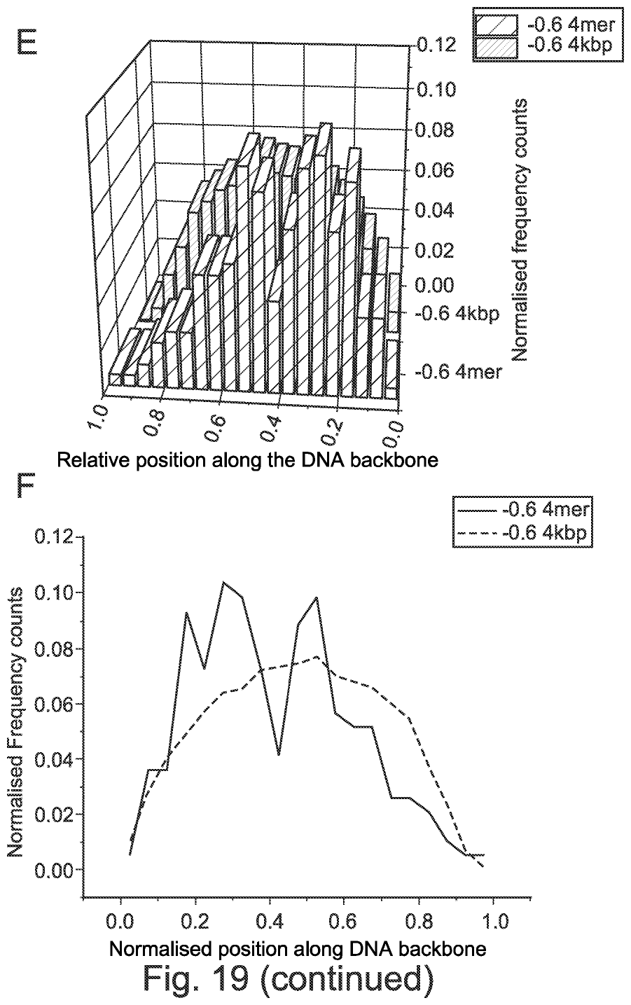

FIG. 19 shows a comparison of sub events analysis (frequency count) conducted between 4mer SCoNE DNA and bare 4 kbp DNA fragments. A comparison between sub event threshold (A) shows that increasing threshold value decreases the number of peaks observable (25-175 pA threshold respectively). A 3D illustration of subevent position along the DNA backbone across a range of biases shows similar positioning of the subevents (B), most notably between −-0.6 and −0.7V (C). The 2D representation re-enforced these conclusions (D). Comparison between 4mer SCoNE DNA and bare 4 kbp DNA fragments shows a lack of additional subevent peaks in the bare DNA samples represented in 3D and 2D views for direct analysis (E and F).

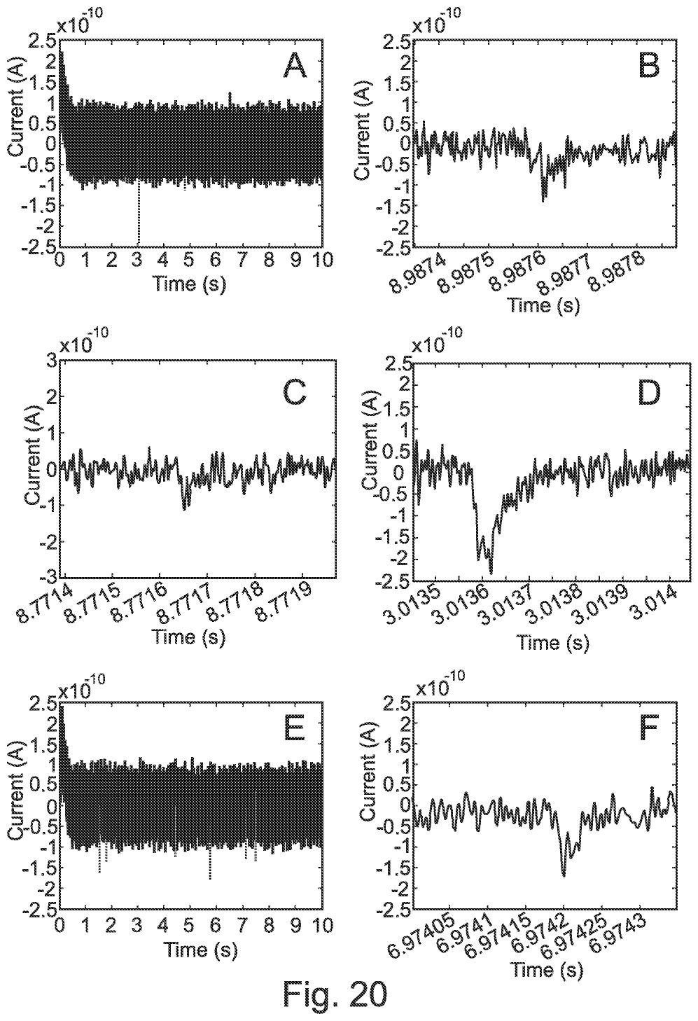

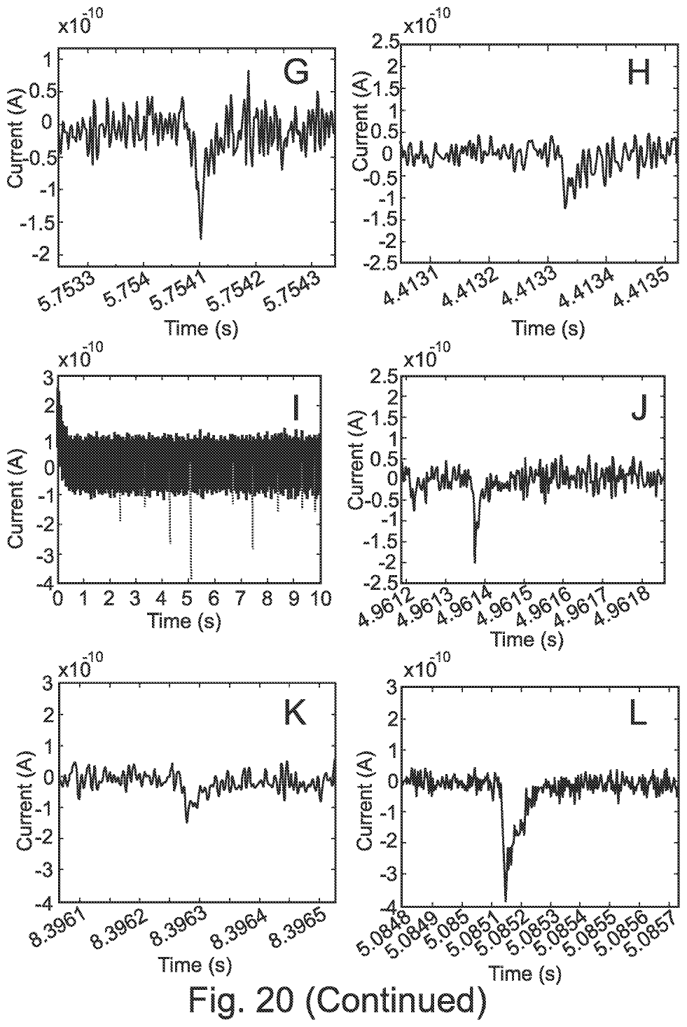

FIG. 20 shows examples of SCoNE DNA current-time traces (A, E and I) for −0.5V, −0.6V, and −0.7V respectively, with single events highlighted for each bias (B-D, F-H, and J-L).

FIG. 121 shows examples of bare 4 kbp DNA current-time traces (A, E and I) for −0.5V, −0.6V, and −0.7V respectively, with single events highlighted for each bias (B-D, F-H, and J-L).

FIG. 1 shows a tip 10 of a probe which is used for obtaining a fluid sample from the oral cavity of a patient. The tip 10 is tapered, and has a shape and size which is selected so that the tip 10 can be used to collect a fluid sample from a gingival crevice. The tip houses a double-barrel nanopipette 12 (a theta pipette) comprising a first barrel 14 and a second barrel 16. The first barrel 14 comprises a first electrode 18, and the second barrel 16 comprises a second electrode 20.

Also located inside the first barrel 14 are carrier molecules 22. For simplicity, only a single carrier molecule is shown in FIG. 1, but it will be appreciated that a plurality of carrier molecules will be present. Each carrier molecule 22 comprises an elongate backbone 24, for example a DNA backbone. Immobilised on the backbone 24 are a plurality of capture moieties 26, which are specific for a target analyte (e.g. a protein biomarker) 28 that is indicative of gingivitis and/or periodontitis.

The fluid sample is taken by applying a voltage to the first electrode 18, which causes fluid comprising the target analytes 28 to flow into the first barrel 14 by electroosmosis (step A). Once inside the first barrel 14, the target analytes 28 are incubated with the carrier molecules 22 for a time sufficient to enable the capture moieties 26 to bind to the target analytes 28 (step B).

Following incubation, the application of a positive bias to the second barrel 16 relative to the first barrel 14 causes translocation of the carrier molecules 22 with capture target analytes 28 from the first barrel 14 to the second barrel 16, causing them to pass through nanopores 30a and 30b therebetween, thereby generating an electrical signal.

FIG. 2 shows modelling results for target binding to a capture probe attached to a DNA carrier, for 12 target proteins. These proteins were identified in samples obtained from patients with different degrees of oral disease, ranging from healthy individuals to individuals with severe periodontal disease. Left axis: experimentally observed target concentrations across the patient population (mean +/− lower and upper bound). Right axis: probability of observing a translocating carrier with the target bound. Simulation parameters: carrier concentration: 10⁻⁹ M; dissociation constant for binding equilibrium: 10⁻¹⁰ M. The plot highlights the relevant concentration ranges (saliva) and provides an estimate of the probability of observing bound targets. It also illustrates how the approach can be employed to determine target concentrations, namely from the observed probability of “bound” events. The latter ranges from 0-100% and can be converted to concentration using the binding constant and the known binding equilibrium.

### EXAMPLE 1

### Nanopipette Fabrication and Characterisation

### Single-Barrel Pipettes

A nanopipette of 20-30 nm diameter may be prepared using the methods described by Loh et al., 2018, Anal Chem. 90, 14063-14071. Briefly, from filamented quartz capillaries (1 mm o.d., 0.5 mm i.d., 7.5 mm in length; Sutter Instruments). The capillaries contain a ˜160 μm glass filament that facilitates the filling of the nanopipette by capillary action. The glass capillaries are first plasma cleaned for 7 minutes (Harrick Scientific) before being loaded into a laser pipet puller (Sutter Instruments).

The inner diameter of the nanopipette can be estimated from the conductance of the pipette in 1 M KCI and/or using transmission electron microscopy (TEM) or optical microscopy.

TEM imaging of the nanopipettes can be carried out using a JEOL JEM-2100F TEM. The measurement of the images can be conducted using ImageJ.61. Sample preparation may be carried out as follows: The tip of the pipet is positioned such that it is sitting parallel to the centre of the Cu TEM slot grid (catalogue no. GG030, Taab Laboratory Equipment Ltd.) and glued to the grid (e.g. using a two-component epoxy glue). The glue is left to set (e.g. for 6 h), after which the pipette attached to the grid is cleaned under UV and ozone for 20 min (UVOCS). It is then sputter coated (Polaron Quorum Technologies) with 10 nm Cr to reduce charging effects. The parts of the pipette lying just outside the grid may be cut off using a scalpel before the grid is placed in the sample holder of the TEM.

### Double-Barrel Pipettes

Double-barrel nanopipettes (also known as “theta pipettes) can be fabricated using the methods described in Saha-Shah et al,. Analyst (2016), 141, 1958-1965 and Saha-Shah et al,. Chem. Sci., 2015,6, 3334-3341. Briefly, theta capillaries are pulled using a laser-based pipette puller (P-2000, Sutter Instrument, Novato, CA) and then a focused ion beam (Zeiss Auriga® Modular Cross Beam workstation Oberkochen, Germany, FIB), followed by milling each barrel to ˜1 μm (internal diameter).

### EXAMPLE 2—OVERVIEW OF A METHOD OF MAKING A MODULAR POLYNUCLEOTIDE

FIG. 3 is a schematic overview of a method of making mature conjugate subunits, which in turn, can be used to create a modular polynucleotide (F) according to the invention. As shown in step (A), a single aminated probe is conjugated to DBCO to create a modified probe for further conjugation. (B) Short, blunt-ended DNA fragments (less than 100 bp in length) are azidated using a methyl transferase (m.Taq1), and an azide modified SAM. (C) Independently, the modified probes and their respective azidated short, blunt-ended DNA fragments are combined together in a copper-free azide-alkyne reaction (a Click-iT™ reaction) to form of immature conjugate subunits. (D) The immature conjugate subunits are then combined into a single vial along with spacer DNA strands (at least 100 bp in length), T5 exonuclease, Taq DNA Ligase, and Taq DNA polymerase. (E) In this step, the 5′ ends of the spacer DNA strands are digested by T5 exonuclease to create sticky ends. In addition, the 5′ ends of the immature conjugate subunits are digested by T5 exonuclease to create mature conjugate subunits (i.e. short, probe bound DNA fragments with sticky ends, which, in this case, are complementary to the sticky ends of the spacer DNA). The T5 exonuclease is unable to navigate across the site at which the probe is attached to the polynucleotide of the immature conjugate and therefore gets knocked off. The spacer DNA strands with sticky ends and the mature conjugate subunits are thus able to form bonds via their matching sticky ends. DNA polymerase fills in any gaps created during digestion and DNA ligase seals the scars, forming phosphodiester bonds, thus resulting in the formation of a modular polynucleotide (a SCoNE structure) shown in (F).

### EXAMPLE 3—PROTOCOL FOR CREATING A MODULAR POLYNUCLEOTIDE

### Materials and Methods

### Stock Concentrations

### 50 μM DBCO-NHS-ester

a) 1 mg of DBCO-NHS-ester added to 1 ml of DMSO (HPLC grade) to create a 2.5 mM stock

b) Take 1 μl of the 2.5 mM added to 49 μl of nuclease free water to create a working stock of 50 μM

### 1. Creating the Modified Probe

a) Each probe should be modified in a separate PCR tube, the volumes provided can be amplified as necessary

b) Pipette 18 μl of 50 mM DBCO-NHS-ester into a PCR grade nuclease free vial

c) Add 2 μl of prepared amine linked capture probe of interest (prepared as indicated by the manufacturer)

d) Incubate at room temperature for a minimum of 2 hours-overnight

e) Modified probes using ssDNA and aptamers are stable at 23° C. for 5 days.

### 2. Creating Modified Probe Strands (Azidation)

a) Each probe strand should be modified in a separate PCR tube, the volumes provided can be amplified as necessary

b) Set up the PCR tube with the following reactants and their respective volume, ensuring that the m.Taq1 enzyme is the final component to be added. Final volume is 20 μl. This can be scaled up dependant on the requirements.

| Reactant | Volume (μl) |
|---|---|
| DNA strand (600 ng/ul) | 1 |
| Cutsmart buffer | 2 |
| (1× buffer comprises: | |
| 50 mM potassium acetate | |
| 20 mM Tris-acetate | |
| 10 mM magnesium acetate | |
| 100 μg/ml BSA | |
| pH 7.9 at 25° C.) | |
| RAdoHcy-8-Hy-PEG-N3 (AW39) (or a | 1 |
| modified SAM molecule containing an | |
| azide group) | |
| m.Taq1 | 0.5 |
| Nuclease free water | 15.5 |

c) Incubate reaction vessel at 40° C. for 2 hours

d) Pipette 0.5 μl of Protinase K (18 mg/ml) into the vial

e) Incubate at 50° C. for 1 hour

f) Allow to cool to room temperature for 20 minutes

g) Perform PCR clean-up protocol (GenElute™ Sigma-Aldrich)
a. Ensure elution is into 20 μl of either elution buffer or nuclease free water
b. Concentration should be determined at this point (e.g. using Nanodrop)

h) Can be stored at 4° C. until required

### 3. Full Construction of Probe Strands

a) Each probe strand should be constructed in a separate PCR tube, the volumes provided can be amplified as necessary.

b) Pipette 4 μl of azidated probe strand into a PCR tube

c) Pipette 2 μl of modified probe into the same tube

d) Incubate at room temperature for a minimum 1.5 hours, can be left overnight.

e) Can be stored at 4° C. until required

### 4. Assembly

a) All components should be added into the same PCR vial for assembly.

b) Pipette equal concentrations of the reagents into a PCR tube to create the IADL-mastermix.

The current set up uses the following volumes due to concentration;

| | Volume to add (ul) | Strand | Volume to add (ul) |
|---|---|---|---|
| Spacer1 | 1.5 | Probe1 | 1 |
| Spacer2 | 2 | Probe2 | 1 |
| Spacer3 | 1.5 | Probe3 | 1 |
| Spacer4 | 2 | Probe4 | 1 |
| Spacer5 | 1.5 | Probe5 | 1 |
| Spacer6 | 2 | Probe6 | 1 |
| Spacer7 | 1.5 | Probe7 | 1 |
| Spacer8 | 1.5 | Probe8 | 1 |
| Spacer9 | 1.5 | Probe9 | 1 |
| Spacer10 | 2 | Probe10 | 1 |

c) Ensure the IADL-mastermix is mixed well using a pipette

d) In a new PCR vial pipette 10 μl of the IADL-mastermix

e) Pipette 5 μl of nuclease free water and mix well

f) Pipette 5 μl of Gibson assembly mastermix [mastermix comprises a T5 exonuclease, a progressive polymerase (such as Taq polymerase) and Taq ligase (New England BioLabs—Gibson Assembly® Master Mix/Gibson Assembly®Cloning Kit-NEB # E2611S/L, # E5510S], and mix well (half of recommended)

g) Incubate at 40 oC for 1.5 hours (can be incubated at this temperature overnight)

h) Desired product has been isolated

### 5. Current Isolation Technique

a) Run the product on a 1% agarose gel at 80V for 80 minutes in 1× TAE buffer

a. The product can now be isolated by size comparison (at approximately 0.5 cm from pipetting well site)

b) Excise the DNA band from the gel using a sterile scalpel

c) Perform Gel extraction (GenElute™ Gel Extraction Kit Sigma-Aldrich)
a. Ensure elution is into 20 μl of either elution buffer or nuclease free water
b. Final concentration can be determined now

d) Product is available for use

### EXAMPLE 4—CONFIRMATION OF MATURE CONJUGATE SYNTHESIS

FIG. 4 is an agarose gel showing an N of 1 SCoNE (Sterically Controlled Nuclease Enhanced DNA Assembly), i.e. the method used to of make a modular double-stranded polynucleotide according to the invention. The gel demonstrates that in lanes 6 and 8 there are bands above the 2 kbp cut off (as indicated by the line using the gene ruler, lane 1, and the 2 kbp fragment, lane 4) illustrating that it is possible to form a dimer (2 probe strands, 2 spacer strands) using the SCoNE technique. This is further shown by the absence of this band in lane 5 (negative control) highlighting that it is the conjugation that allows the formation of the larger structures. The lower band at 1 kbp is unreacted spacer DNA.

FIG. 5 is an agarose gel showing an N of 2 SCoNE experiments. The gel demonstrates it is possible to generate a dimer (2 probe strands, 2 spacer strands) using the scone technique, as highlighted in lanes 5 and 8. The absence of a fragment above 2 kbp in lane 7 highlights that it is the use of the conjugates which allows for these fragments to be formed. The 2 kbp cut-off height is indicated by the line using the gene ruler, lane 1, and the 2 kbp fragment, lane 3. The lower band at 1 kbp is unreacted spacer DNA.

FIG. 6 is an agarose gel showing an N of 3 and 4 SCoNE experiments. Lanes 2 and 3 (N3 and N4 respectively) highlight the formation of the dimer (2 probe strands, 2 spacer strands). The absence of these fragments in the negative controls in lanes 4 and 5 (N3 and N4 respectively) highlight that it is the conjugation of the probes which allow for these fragments to be formed. The 2 kbp cutoff height is indicated by the line using the gene ruler, lane 1, and the 2 kbp fragment, lane 8. The lower band at 1 kbp is unreacted spacer DNA.

FIG. 9 is an agarose gel showing several intermediate steps are generated during the reaction (lanes 6 and 7, 18.2 and 18.3), which indicates the depletion of the starting DNA. The absence of 1 kbp DNA (lanes 6 and 7, B.4) indicates that the spacer DNA has been fully incorporated into SCoNE structures unlike in the negative control (lane 8, B.5) where the 1 kbp fragments are still present.

### EXAMPLE 5

As described above, the inventors have shown that they are able to create structures with the expected weight. This was shown in 1% agarose gels.

This example relates to isolation. The nomenclature provided in the table immediately below is relevant. In this example, experiments were performed to increase yield of single product collection, removing unwanted DNA fragments from the initial one pot reaction mixture, and to determine the most effective positioning of the biotin groups to allow for this.

| Nomenclature | Explanation |
|---|---|
| S | Spacer DNA strand (1000 bp) |
| P | Probe DNA strand with aptamer attached |
| | (60 bp) |
| B | Probe DNA strand with biotin group attached |
| | (60 bp) |
| 3mer | A SCoNE structure comprised of 3S and 3P |
| | or B strands |
| 4mer | A SCoNE structure comprised of 4S and 4P |
| | or B strands |
| 3S, 1P₃, 2B_{1, 2} | Indicates 3 S strands, 1 P strand at the third |
| | position, and 2 B strands at positions 1 and 2 |

The inventors have included an additional probe structure (DBCO-dPEG®12-biotin, Sigma-Aldrich) to assist with isolation. The biotin groups added to the SCoNE structure were tested for their position effectiveness and the effectiveness of their use as a purification method. The inventors found that positions 1 and 2 of the final structure provided the highest yield.

### Isolation

First, the inventors directly tested their ability to create the new biotin structures and tested their isolation method for these. Lane gaps between samples and the ruler were added to increase resolution of the low concentration samples as shown in FIG. 10.

From these results the inventors were able to show they can create p strands containing biotin and extract them from starting materials prior to the assembly step.

The positioning of the biotin group within the SCoNE structure was then compared with the achieved yield. Due to working in very low concentrations it was difficult to use gel electrophoresis for complete quantitation, however Gray values were compared across all groups tested as shown in FIG. 11.

From these results, the inventors were able to show that they can extract assembled SCoNE structures using the biotin tag they added in from the mix of starting elements. As is shown in FIG. 12, the inventors also demonstrated that the positioning of the biotin group is also important for extraction.

This difference shows that biotin at positions 1 and 2 provide the highest efficiency in extraction. This could be due to multiple bindings to streptavidin during extraction assisting the binding of the SCoNE structure. The inventors observed a decrease in binding of 2.60%, 2.88%, 1.21%, and 2.48% respective to 3S,1P₃,2B_{1,2}.

### Testing Binding Capacity

To perform binding capacities and efficiency of final structure formation, one standard (A) and three custom designed (B-D) ELISAs were performed, illustrated in FIG. 13. Several wash steps were performed between steps.

### Notes on Extraction and Utilisation of SCoNE Structures

Scone structures are generated at different concentrations dependant on the vial used, therefore data is normalised to expected SCoNE concentration. Nanodrop is used to determine starting SCoNE concentration. Through calculating yields of SCoNE structures through the gel experiments, 3mer and 4mer structures were generated with efficiencies of 57-61%. Some of the SCoNE structures are also lost during extraction, this loss is approximately 20%. As the inventors extract twice, this is taken into account during calculations. Where applicable to bind the biotin groups on the SCoNE structure, an excess of 300%, to the expected concentration, streptavidin was used and incubated for 30 minutes prior to experiments taking place. SCoNE structures were incubated for 30 minutes with the respective protein/s at 0.5 ng/ml. SCoNE structures from different assembly reactions were used for each experiment to ensure reliability in the assembly and isolation protocols. All experiments were completed to N=6. Expected concentrations were 54.45, 54.49, 21.50, and 13.82 ng/ml (for I L6 run 2, procalcitonin, and no I L6 experiments).

### Results and Discussion

From the results of the ELISA performed, the inventors showed that they were able to successfully isolate SCoNE structures with functional capture groups. Results from the ELISAs are split into the 3mer and 4mer experiments. With respect to the starting concentrations expected, the yield of producing and successfully binding the proteins of interest are 71.31% (ELISA A), 67.49% (ELISA B), 54.00% (ELISA C), 74.88% (ELISA C), and 65.91%(ELISA C, using the reverse antibody set; IL6 primary with procalcitonin secondary). When removing the I L6 protein for ELISA D, a yield of 1.80% was observed. This indicates that not all biotin sites were fully occupied, therefore an error of 1.8% can be applied to all values obtained, bar ELISA B experiments.

### EXAMPLE 6

This example relates to translocation. Translocation of bound SCoNE DNA through a nanopore was performed to assess firstly, the ability of the sensing apparatus to accurately detect DNA translocation. Secondly, to categorise the profile of SCoNE DNA and its differences from that of bare DNA. Finally, inclusion of a bound probe was utilised to determine the stability of the probe binding, translocation potential under real experimental conditions, and develop data analysis tools for subsequent comparison. The inventors successfully detected SCoNE DNA with bound analyte and successfully determined its differences to that of bare DNA.

### Methods

Cleaned amber liquid cells were filled with 2 ml of the 4 M LiCI 10% TE solution. SCoNE DNA was added into the vial to achieve a final concentration of approximately 80 μM. A size-determined nanopipette was inserted into the cell, submerging the tip in the liquid. Anodized silver/silver chloride electrodes, soldered to gold contact pins, were added to the setup, such that one electrode sat inside the pipette chamber, and the other in the bulk solution, outside of the pipette. This was then attached to a custom low noise amplifier, sampling at 1 MHz. A 100 kHz in-line filter was attached to the output of the amplifier, and connected directly to a Picoscope 4262 oscilloscope, which was used for real-time monitoring of the system. A custom MATLAB script was used to control the bias voltage applied to the system, which allowed for changing the input voltage during measurements. Each scan was saved for further event and sub event analysis performed by custom MATLAB scripts.

### Translocation Results

When translocating the 4mer structure, as indicated in FIG. 15, the inventors observed translocation of the 4mer structure as illustrated in FIG. 16.

The inventors also showed that translocation follows the expected trend (to bare DNA) with increasing bias directly correlated with a decrease in event duration, FIG. 17.

When comparing average translocation profiles, see FIG. 18, the inventors observed that whilst the SCoNE translocation events have significant current decreases at specific points along the translocation, the bare DNA has less structure to where sub events can occur. It was also observed that decreases in current are matched by similar returns to the positive in bare DNA, these positive spikes are not observed in SCoNE DNA. This is further highlighted when looking at singular events.

Sub event analysis provides a further insight into the substructure of the events as highlighted in FIG. 19. The inventors applied a threshold for determining sub event analysis of between (10.A). The DNA structure is approximately 4.1 kbp long, and comparisons are made between SCoNE 4mer and 4 kbp DNA fragments (NoLimits). DNA has the capability to translocate both forwards and backwards through the nanopore. In several of the figures herein, the size of the backbone has been normalised to values 0-1 with the relative positions of sub structures falling between these values. In a forwards translocation, the inventors expected to see peaks at positions near 0, 0.25, 0.5 and 0.75. In a backwards translocation, the inventors expected to see peaks at positions 0.25, 0.5, 0.75 and near 1. As can be seen from the from FIG. 19A, there are several peaks which emerge from the events generated. In lower thresholds (25-75 pA) the inventors observed peaks at near 0, near 0.25, 0.5, near and some emergence near 1. Due to the size of the biotin binding groups, and the unbound aptamer, it is possible that during data acquisition some of the resolution near the beginning and end of the event is lost. When the inventors applied a threshold between 100-150 pA, the inventors observed loss of these initial, and ending peaks, however obtained a greater resolution of subevents at positions 0.25 and 0.5. Above 150 pA threshold the inventors observed only the peak at 0.25 remaining, indicating they had filtered out useful subevents. When the inventors use a 100 pA and visualise the event across the different biases applied (see FIG. 19B) they observed that there are clear peaks across the datasets at positions 0.5, and potentially near 1. This is even visible in the 0.5V bias which had a very limited number of translocations. These peaks are most notable in the −0.6 and −0.7V biases (see FIGS. 19C, and 19D). When the inventors compared the subevent analysis to bare 4 kbp DNA (nearest comparable size), they observed that the bare DNA creates a smooth shape across the event, lacking notable subevent peaks across the backbone (see FIGS. 19.E and 19.F). Due to differences in starting concentration, frequency counts have been normalised against total counts for direct comparison.

### Single Event View

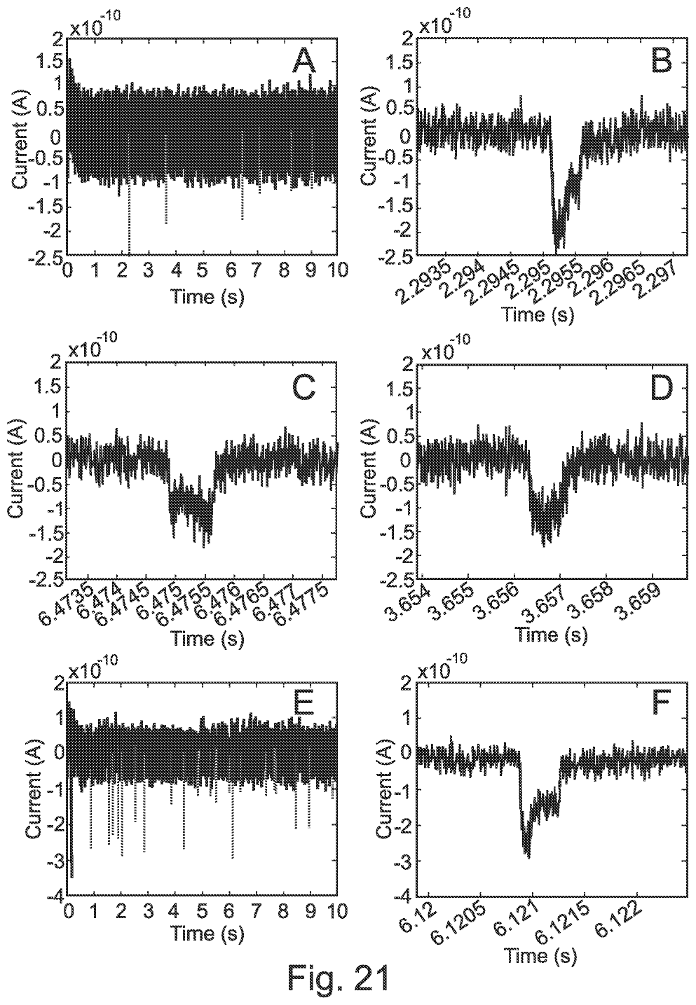

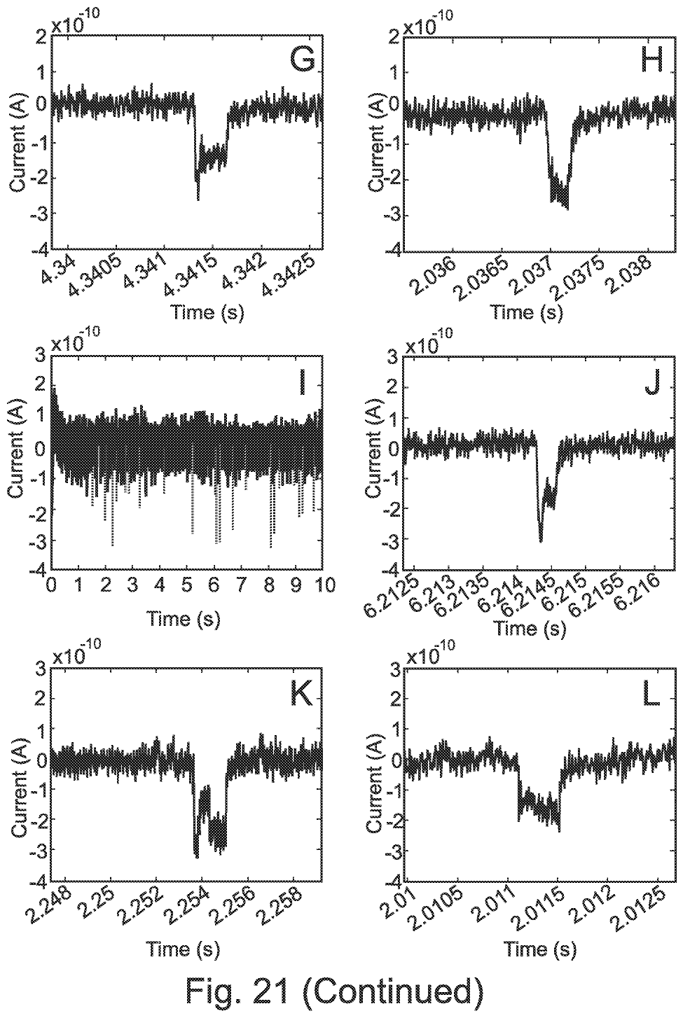

When the inventors viewed singular events, they observed the trends observed in both the average analysis and the subevent analysis. FIG. 20 highlights a few events (excluding A, E and I) observed in the scans (A, E and I) across three biases used during experiments (-−0.6V and −0.7V respectively). These show a clear definition of sub events appearing from the baseline as opposed to noise contribution. This can be determined by comparison to bare 4 kbp DNA translocation events (FIG. 21, excluding A,E and I). Experiments were conducted using nanopores of similar size (usually between 10-20 nm pores, depending on the size of the analyte), with the only difference being the DNA in solution. It can also be noted that translocation frequency is much higher in bare 4 kbp, this is due to a higher starting concentration.

### SUMMARY

Overall, the inventors have:
demonstrated that SCoNE DNA can be translocated and sub-events can be detected successfully;
demonstrated that SCoNE DNA-protein binding can be successfully achieved;
developed a DNA extraction method and tested this successfully;
developed new ELISA methods and tested these for protein binding with high efficiency; and
compared SCoNE to bare 4 kbp DNA and shown a clear distinction between event shapes

### EXAMPLE 7

The invention further includes the subject matter of the following numbered paragraphs (paras).
1. A method of making an immature conjugate subunit, the method comprising:
   conjugating a (first) probe, for binding a (first) analyte, to a probe conjugation site of a (first) double-stranded polynucleotide, to create a (first) immature conjugate subunit.
2. A method of making a mature conjugate subunit, the method comprising:
   conjugating a (first) probe, for binding a (first) analyte, to a probe conjugation site of a (first) double-stranded polynucleotide, to create a (first) immature conjugate subunit; and
   forming a (first) mature conjugate subunit by cleaving the double-stranded polynucleotide of the (first) immature conjugate subunit to form a first sticky end, or a first sticky end and/or a second sticky end.
3. The method according to paragraph 2, further comprising:
   annealing the first sticky end or the second sticky end of the (first) mature conjugate subunit to a complementary sticky end of a (first) double-stranded polynucleotide spacer.
4. The method according to any one of paragraphs 1 to 3, wherein conjugating comprises contacting a polynucleotide having a probe conjugation site functionalised with an azide group with a probe functionalised with a DBCO-NHS ester.
5. The method according to any one of paragraphs 1 to 4, wherein the polynucleotide of the immature conjugate subunit comprises a first blunt end and/or a second blunt end.
6. The method according to any one of paragraphs 2 to 5, wherein the polynucleotide of the double-stranded mature conjugate subunit is between about 20 and about 100 base pairs/nucleotides in length.
7. The method according to any one of paragraphs 2 to 6, wherein the double-stranded polynucleotide of the mature conjugate subunit comprises 5′-sticky ends and/or 3′-sticky ends.
8. The method according to any one of paragraphs 2 to 7, wherein the mature conjugate subunit is formed by cleaving the double-stranded polynucleotide of the immature conjugate subunit with an enzyme, optionally wherein the enzyme is a 5′ exonuclease, such as a T5 exonuclease.
9. The method according to any one of paragraphs 3 to 8, wherein annealing comprises contacting a (first) mature conjugate subunit with the (first) double-stranded polynucleotide spacer in the presence of a DNA ligase and a DNA polymerase.
10. A method of making a modular polynucleotide comprising:
   i. creating a mature conjugate subunit according to the method of any one of paragraphs 2 to 9;
   ii. repeating the method of any one of paragraphs 2 to 9 to create a total of two or more mature conjugate subunits,
   wherein each of the two or more mature conjugate subunits has a (first or second) sticky end that is complementary with a (first or second) sticky end of a separate mature conjugate subunit; and
   iii. annealing the complementary sticky ends of the two or more mature conjugate subunits to create a modular polynucleotide comprising a double-stranded polynucleotide backbone having two or more mature conjugate subunits.
11. The method according to paragraph 10, wherein annealing comprises contacting the two or more mature conjugate subunits in the presence of a DNA ligase and a DNA polymerase.
12. The method according to paragraph 9 or 11, wherein the DNA ligase is Taq ligase.
13. The method according to paragraph 9, 11 or 12, wherein the DNA polymerase is Taq DNA polymerase.
14. An immature conjugate subunit comprising:
   a probe, for binding an analyte, conjugated to a probe conjugation site of a double-stranded polynucleotide,
   wherein the probe conjugation site comprises a nucleotide sequence specific for a transferase enzyme.
15. The immature conjugate subunit according to paragraph 14, wherein the polynucleotide of the double-stranded mature conjugate subunit is between about 20 and about 100 base pairs/nucleotides in length.
16. The immature conjugate subunit according to paragraph 14 or paragraph 15, wherein the polynucleotide of the double-stranded mature conjugate subunit comprises a first blunt end or a second blunt end, and/or wherein the polynucleotide of the double-stranded mature conjugate subunit comprises 5′-sticky ends or 3′-sticky ends.
17. A double-stranded modular polynucleotide comprising: a double-stranded polynucleotide backbone comprising a plurality of probe conjugation sites, each site having a nucleotide sequence specific for a transferase enzyme, wherein each probe conjugation sites is separated by at least 16 base pairs, and wherein at least one probe conjugation site is conjugated to a single probe for binding an analyte.
18. The immature conjugate according to any one of paragraphs 14 to 16 or the double-stranded modular polynucleotide according to paragraph 17, wherein the transferase enzyme is a methyl transferase, or wherein the nucleotide sequence specific for a transferase enzyme comprises a CpG island or a “TOGA” nucleotide sequence.
19. The double-stranded modular polynucleotide according to paragraph 17 or paragraph 18, wherein each of the probe conjugation sites is separated by at least about 30 base pairs.
20. The The method of any one of paragraphs 2 to 13, or the immature conjugate subunit according to any one of paragraphs 14 to 16, or the double-stranded modular polynucleotide according to any one of paragraphs 17 to 19, wherein the probe(s) is/are one or more selected from the group consisting of a single-stranded nucleotide, an antibody, a (functional) fragment of an antibody and/or an aptamer.
21. A kit for determining if one or more analyte(s) is/are present in a sample, the kit comprising:
   i. a double-stranded polynucleotide spacer; and
   ii. an immature conjugate subunit according to any one of paragraphs 14 to 16 or an immature conjugate subunit made by the method of paragraph 1; or
   iii. a modular polynucleotide according to any one of paragraphs 17 to 20.
22. A kit for diagnosing a test subject suffering from a medical condition, the kit comprising:
   i. a double-stranded polynucleotide spacer; and
   ii. an immature conjugate subunit according to any one of paragraphs 14 to 16 or an immature conjugate subunit made by the method of paragraph 1; or
   iii. a modular polynucleotide according to any one of paragraphs 17 to 20,
   wherein the presence of one or more analyte(s) in a bodily sample from a test subject is indicative that the subject suffers from the medical condition, or wherein the absence of the one or more analyte(s) from a bodily sample from a test subject is indicative that the subject suffers from the medical condition.
23. The kit of paragraph 21 or 22, wherein the kit comprises a negative control and/or a positive control.
24. The kit of any one of paragraphs 21 to 23, wherein the kit comprises a buffer and/or one or more enzymes selected from the group consisting of a DNA polymerase, a nuclease (e.g. exonuclease) and a DNA ligase.
25. The A method of determining if one or more analyte(s) is/are present in a sample, the method comprising:
   i. contacting a modular polynucleotide according to any one of paragraphs 17 to 20 with a test sample; and then
   ii. analysing the modular polynucleotide using a carrier enhanced-resistance pulse sensing technology to determine if one or more analyte(s) is/are present in the test sample.

1. A system for diagnosing an oral disease in a subject, wherein the system comprises:
a probe which is configured to collect a fluid sample from an oral cavity of the subject; and
a detector which is configured to detect in the fluid sample at least one of a presence and a concentration of an analyte which is indicative of the oral disease, wherein the detector is configured to detect at least one of the presence and the concentration of the analyte using resistive pulse sensing.

2. The system of claim 1, wherein the detector is in fluid communication with the probe.

3. The system of claim 1, wherein the detector and the probe are integrated into a single device.

4. The system of claim 1, wherein the probe comprises a tip which is configured to collect the fluid sample via at least one of suction, capillary action, and electroosmosis.

5. The system of claim 4, wherein the tip is configured for collecting gingival crevicular fluid (GCF) from a gingival crevice.

6. The system of claim 1, wherein the probe is a periodontal probe comprising a hollow tip.

7. The system of claim 1, wherein the detector comprises a nanopipette.

8. The system of claim 7, wherein the nanopipette is housed within the tip of the probe.

9. (canceled)

10. A composition comprising a plurality of carrier molecules for detecting of a target analyte using resistive pulse sensing, wherein each of the carrier molecules is functionalized with a capture moiety, wherein the capture moiety is capable of specifically binding to a biomarker of an oral disease.

11. The composition of claim 10, wherein the carrier molecules are nucleic acids (c.g. DNA).

12. The composition of claim 10, wherein the capture moiety is an antibody.

13. (canceled)

14. (canceled)

15. (canceled)

16. (canceled)

17. A method of diagnosing an oral disease in a subject, the method comprising using resistive pulse sensing to detect at least one of athe presence and a concentration of a target analyte in a fluid sample obtained from an oral cavity of a subject.

18. The method of claim 17, further comprising:
a) applying a voltage across a nanopore to effect a translocation of any molecules present in the fluid sample through the nanopore;
b) detecting a signal generated by the translocation of the molecules through the nanopore; and
c) optionally, comparing the signal detected with a reference signal.

19. The method of claim 18, further comprising incubating the fluid sample with a plurality of carrier molecules prior to translocation, wherein the carrier molecules are functionalized with a capture moiety which is capable of specifically binding to the target analyte.

20. The method of claim 17, wherein the method is carried out using a probe configured for collecting the fluid sample from the oral cavity of the subject, wherein the probe comprises a hollow tip in which is housed a nanopipette.

21. The method of claim 20, wherein the probe is a periodontal probe.

22. The method of claims 17, wherein the fluid sample is selected from the group consisting of saliva andef gingival crevicular fluid (GCF).

23. The system method of claim 17, wherein the oral disease is periodontal disease.

24. The system of claim 1, wherein the detector is coupled to the probe.

25. The system of claim 1, wherein the oral disease is periodontal disease.
